(19) United States
(12) Patent Application Publication
(10) Pub. No.: US 2009/0018613 A1
(43) Pub. Date: Jan. 15, 2009

(54) REGULATION OF VASCULAR ENDOTHELIAL GROWTH FACTOR (VEGF) GENE EXPRESSION IN TISSUE VIA THE APPLICATION OF ELECTRIC AND/OR ELECTROMAGNETIC FIELDS

(75) Inventor: Carl T. Brighton, Malvern, PA (US)

Correspondence Address: GIBSON & DERNIER L.L.P., 900 ROUTE 9 NORTH, SUITE 504, WOODBRIDGE, NJ, 07095, US

(73) Assignee: GENESTIM, LLC, Short Hills, NJ (US)

(21) Appl. No.: 11/832,790

(22) Filed: Aug. 2, 2007

Related U.S. Application Data
(60) Provisional application No. 60/821,211, filed on Aug. 2, 2006.

(51) Int. Cl. A61N 1/05

(52) U.S. Cl. 607/50

(57) ABSTRACT

Methods and devices for the regulation of gene expression in tissue by applying an electric and/or electromagnetic field generated by specific and selective signals so as to treat diseases, conditions, and/or tissue. Gene expression is the up-regulation or down-regulation of the process whereby specific portions (genes) of the human genome (DNA) are transcribed into mRNA and subsequently translated into protein. Methods and devices are described for the regulation of Vascular Endothelial Growth Factor (VEGF) protein gene expression in endothelial cells of various targeted tissues via the capacitive coupling or inductive coupling (e.g., by electrodes or one or more coils or other field generating devices disposed with respect to the targeted cells) of specific and selective signals to the cells of these tissues, where the resultant electric and/or electromagnetic fields treat diseased or injured tissues. The resulting methods and devices are useful for the targeted treatment of peripheral vascular disease, cardiovascular disease, macular degeneration, wound healing, tendon and ligament healing, in preventing tumor growth or spread, and other conditions in which VEGF protein may be implicated.

### CROSS-REFERENCE TO RELATED APPLICATIONS

This application is based on and claims the benefit of U.S. Provisional Patent Application No. 60/821,211, filed Aug. 2, 2006, the entire disclosure of which is hereby incorporated by reference.

### BACKGROUND OF THE INVENTION

The present invention is directed generally to methods of regulating gene expression in tissue (e.g., injured or diseased tissue) by applying to such tissue electric and/or electromagnetic fields generated by specific and selective signals, for treating such tissue, as well as to devices for generating such fields. The present invention is directed particularly to methods of regulating expression of vascular endothelial growth factor (VEGF) in tissue (e.g., injured or diseased tissue) by applying to such tissue electric and/or electromagnetic fields generated by specific and selective signals, for treating such tissue, as well as to devices for generating such fields.

The bioelectrical interactions and activity believed to be present in a variety of biological tissues are one of the least understood of the physiological processes. However, there has recently been much research into these interactions and activity regarding the growth and repair of certain tissues. In particular, there has been much research into stimulation by electric and/or electromagnetic fields and its effect on the growth and repair of bone and cartilage, and on the regulation of the expression of various growth factors. Researchers believe that such research may be useful in the development of new treatments for a variety of medical problems.

Osteoarthritis, also known as degenerative joint disease, is characterized by degeneration of articular cartilage as well as proliferation and remodeling of sub-chondral bone. The usual symptoms are stiffness, limitation of motion, and pain. Osteoarthritis is the most common form of arthritis, and prevalence rates increase markedly with age. It has been shown that elderly patients with self-reported osteoarthritis visit doctors twice as frequently as their unaffected peers. Such patients also experience more days of restricted activity and bed confinement compared to others in their age group. In one study, the majority of symptomatic patients became significantly disabled during an 8-year follow-up period. Massardo et al., Ann Rheum Dis 48: 893-7 (1989).

Non-steroidal anti-inflammatory drugs (NSAIDs) remain the primary treatment modality for osteoarthritis. It is unknown whether the efficacy of NSAIDs is dependent upon their analgesic or anti-inflammatory properties or the slowing of degenerative processes in the cartilage. There is also a concern that NSAIDs may be deleterious to patients. For example, NSAIDs have well known toxic effects in the stomach, gastrointestinal tract, liver, and kidney. However, aspirin inhibits proteoglycan synthesis and normal cartilaginous repair processes in animals. One study in humans suggested that indomethacin might accelerate breakdown of hip cartilage. All adverse effects appear more commonly in the elderly—the very population most susceptible to osteoarthritis.

In the disease commonly known as osteoporosis, bone demineralizes and becomes abnormally rarefied. Bone comprises an organic component of cells and matrix as well as an inorganic or mineral component. The cells and matrix comprise a framework of collagenous fibers that is impregnated with the mineral component of calcium phosphate (85%) and calcium carbonate (10%) that imparts rigidity to the bone. While osteoporosis is generally thought to afflict the elderly, certain types of osteoporosis may affect persons of all ages whose bones are not subject to functional stress. In such cases, patients may experience a significant loss of cortical and cancellous bone during prolonged periods of immobilization. Elderly patients are known to experience bone loss due to disuse when immobilized after fracture of a bone, which may ultimately lead to a secondary fracture in an already osteoporotic skeleton. Diminished bone density may lead to vertebrae collapse, fractures of hips, lower arms, wrists, and ankles, as well as to incapacitating pains. Alternative non-surgical therapies for such diseases are needed.

Vascular endothelial growth factor (VEGF) induces endothelial cell proliferation, promotes cell migration, inhibits apoptosis, induces vasculogenesis, promotes angiogenesis, and, if inhibited, abrogates the development and spread of a wide variety of tumors (Neufeld, G., et al, FASEB Journal, 13: 9-22, 1999). VEGF is also expressed in a variety of cells in response to various soluble mediators, such as cytokines and other growth factors, and environmental factors, such as hypoxia (Harry, L. E. and Paleolog, E. M. Birth Defects Research (Part C) 69: 363-374, 2003). Gene regulation in endothelial cells by physical forces, both mechanical and electrical, has also been demonstrated. Cyclic strain regulates the production of a variety of vasoactive substances by endothelial cells (Oluwole, B. O., et al, Endothelium, 5: 85-93). VEGF gene expression is up-regulated in chronically stimulated skeletal muscle in rats via implanted electrodes delivering 10 Hz, 300 μs pulses (Hang, J., et al, Rapid Communication, American Physiological Soc., H1827-H1831 1995). Recently it was shown that direct current applied to endothelial cells in culture stimulated VEGF production, resulting in cellular reorientation, elongation, and migration (Zhao, M., et al, J. of Cell Science, 117: 397-405, 2004).

Pulsed electromagnetic fields (PEMF) and capacitive coupling (CC) have been used widely to treat non-healing fractures (nonunion) and related problems in bone healing since approval by the Food and Drug Administration in 1979. The original basis for the trial of this form of therapy was the observation that physical stress on bone causes the appearance of tiny electric currents that, along with mechanical strain, were thought to be the mechanisms underlying transduction of the physical stresses into a signal that promotes bone formation. Along with direct electric field stimulation that was successful in the treatment of nonunion, noninvasive technologies using PEMF and CC (where the electrodes are placed on the skin in the treatment zone) were also found to be effective. PEMFs generate small, induced currents (Faraday currents) in the highly-conductive extracellular fluid, while CC directly causes currents in the tissues; both PEMFs and CC thereby mimic endogenous electrical currents.

The endogenous electrical currents, originally thought to be due to phenomena occurring at the surface of crystals in the bone, have been shown to be due primarily to movement of fluid containing electrolytes in channels of the bone containing organic constituents with fixed negative charges, generating what are called “streaming potentials.” Studies of electrical phenomena in bone have demonstrated a mechanical-electrical transduction mechanism that appears when bone is mechanically compressed, causing movement of fluid and electrolytes over the surface of fixed negative charges in the proteoglycans and collagen in the bone matrix. These streaming potentials serve a purpose in bone, and, along with mechanical strain, lead to signal transduction that is capable of stimulating bone cell synthesis of a calcifiable matrix, and, hence, the formation of bone. Studies of electrical phenomena in cartilage have demonstrated a mechanical-electrical transduction mechanism that resembles those described in bone, appearing when cartilage is mechanically compressed, causing movement of fluid and electrolytes over the surface of fixed negative charges in the proteoglycans and collagen in the cartilage matrix. These streaming potentials serve a purpose in cartilage similar to that in bone, and, along with mechanical strain, lead to signal transduction that is capable of stimulating chondrocyte synthesis of matrix components.

The main application of direct current, CC, and PEMFs has been in orthopedics in healing of nonunion bone fractures (Brighton et al., J. Bone Joint Surg. 63: 2-13, 1981; Brighton and Pollack, J. Bone Joint Surg. 67: 577-585, 1985; Bassett et al., Crit. Rev. Biomed. Eng. 17: 451-529, 1989; Bassett et al., JAMA 247: 623-628, 1982). Clinical responses have been reported in avascular necrosis of hips in adults and Legg-Perthes's disease in children (Bassett et al., Clin. Orthop. 246: 172-176, 1989; Aaron et al., Clin. Orthop. 249: 209-218, 1989; Harrison et al., J. Pediatr. Orthop. 4: 579-584, 1984). It has also been shown that PEMFs (Mooney, Spine 15: 708-712, 1990) and CC (Goodwin, Brighton et al., Spine 24: 1349-1356, 1999) can significantly increase the success rate of lumbar fusions. There are also reports of augmentation of peripheral nerve regeneration and function and promotion of angiogenesis (Bassett, Bioessays 6: 36-42, 1987). Patients with persistent rotator cuff tendonitis refractory to steroid injection and other conventional measures, showed significant benefit compared with placebo-treated patients (Binder et al., Lancet 695-698, 1984). Finally, Brighton et al. have shown in rats the ability of an appropriate CC electric field generated by electric signals to both prevent and reverse vertebral osteoporosis in the lumbar spine (Brighton et al., J. Orthop. Res. 6: 676-684, 1988; Brighton et al., J. Bone Joint Surg. 71: 228-236, 1989).

More recently, research in this area has focused on the effects stimulation has on tissues. For example, it has been conjectured that direct currents do not penetrate cellular membranes, and that control is achieved via extracellular matrix differentiation (Grodzinsky, Crit. Rev. Biomed. Eng. 9:133-199, 1983). In contrast to direct currents, it has been reported that PEMFs can penetrate cell membranes and either stimulate them or directly affect intracellular organelles. An examination of the effect of PEMFs on extracellular matrices and in vivo endochondral ossification found increased synthesis of cartilage molecules and maturation of bone trabeculae (Aaron et al., J. Bone Miner. Res. 4: 227-233, 1989). More recently, Lorich et al. (Clin. Orthop. Related Res. 350: 246-256, 1998) and Brighton et al. (J. Bone Joint Surg. 83-A, 1514-1523, 2001) reported that signal transduction of a capacitively coupled electric signal is via voltage gated calcium channels, whereas signal transduction of PEMFs or combined electromagnetic fields is via the release of calcium from intracellular stores. In all three types of electrical stimulation there is an increase in cytosolic calcium with a subsequent increase in activated (cytoskeletal) calmodulin.

Much research has been directed at studying tissue culture in order to understand the mechanisms of response. In one study, it was found that electric fields increased [³H]-thymidine incorporation into the DNA of chondrocytes, supporting the notion that Na and Ca²⁺ fluxes generated by electrical stimulation trigger DNA synthesis. Rodan et al., Science 199: 690-692 (1978). Studies have found changes in the second messenger, cAMP, and cytoskeletal rearrangements due to electrical perturbations. Ryaby et al., Trans. BRAGS 6: (1986); Jones et al., Trans. BRAGS 6: 51 (1986); Brighton and Townsend, J. Orthop. Res. 6: 552-558, 1988. Other studies have found effects on glycosaminoglycan, sulphation, hyaluronic acid, lysozyme activity and polypeptide sequences. Norton et al., J. Orthop. Res. 6: 685-689 (1988); Goodman et al., Proc. Natl. Acad. Sci. USA 85: 3928-3932 (1988).

It was reported in 1996 by the present inventor that a cyclic biaxial 0.17% mechanical strain produces a significant increase in TGF-β₁ mRNA in cultured MC3T3-E1 bone cells in a cooper dish (Brighton et al., Biochem. Biophys. Res. Commun. 229: 449-453, 1996). Several significant studies followed in 1997. In one study it was reported that the same cyclic biaxial 0.17% mechanical strain produced a significant increase in PDGF-A mRNA in similar bone cells (Brighton et al., Biochem. Biophys. Res. Commun. 43: 339-346, 1997). It was also reported that a 60 kHz capacitively coupled electric field of 20 mV/cm produced a significant increase in TGF-β₁ in similar bone cells in a cooper dish (Brighton et al., Biochem. Biophys. Res. Commun. 237: 225-229, 1997). However, the effect such a field would have on other genes within the body has not been reported in the literature. U.S. patent application Ser. No. 10/257,126, filed Oct. 8, 2002; PCT/US01/05991, filed Feb. 23, 2001; and U.S. Provisional Patent Application Ser. No. 60/184,491, filed Feb. 23, 2000 are incorporated by reference herein.

There is a great need for methods and devices for treating tissue (e.g., diseased or injured tissue), as well as for treating diseases (e.g., osteoarthritis, osteoporosis, cancer, and other diseases). In particular, there is a need for methods and devices that treat tissue and/or diseases by selectively up-regulating or down-regulating the expression of certain genes. More particularly, there is a need for methods and devices that apply treatments (e.g., for peripheral vascular disease, cardiovascular disease, macular degeneration, wound healing, tendon and ligament healing, rheumatoid arthritis, bone healing (e.g., fresh fractures, fractures at risk, delayed healing and nonunion, bone defects, spine fusion, and as an adjunct in any of the above), and/or osteonecrosis), and/or that prevent tumor growth and spread, by selectively regulating the expression of VEGF. The present invention is directed to these, as well as other, important needs in the art.

### SUMMARY OF THE INVENTION

The present invention relates to regulating the expression of genes in tissue by applying to such tissue electric and/or electromagnetic fields generated by specific and selective signals. In particular, the present invention relates to methods of regulating the expression of genes in tissue by applying such fields to such tissue, and to devices employing such methods.

In an embodiment of the invention, a method is provided for treating tissue (e.g., injured or diseased tissue), and/or for treating diseases or other conditions (e.g., peripheral vascular disease, osteoarthritis, osteoporosis, cancer, and/or other diseases or conditions), such method preferably includes the steps of (1) providing electric and/or electromagnetic fields that regulate gene expression in targeted tissue, which fields are generated by specific and selective signals, and (2) exposing such targeted tissue to such fields so as to regulate gene expression therein. As contemplated by the invention, a “specific and selective” signal is preferably a signal that (a) has predetermined characteristics (such as, for example but not limited to, amplitude, duration, duty-cycle, frequency, and/or waveform) such that the signal preferably creates an electric and/or electromagnetic field that will up-regulate or down-regulate expression(s) of a targeted gene or targeted functionally complementary genes (e.g., the specificity of the signal is established by which and how many characteristics the signal has and by the predetermined settings of those characteristics), and (b) can preferably be chosen to create such an electric and/or electromagnetic field that will up-regulate or down-regulate expression(s) of a targeted gene or targeted functionally complementary genes in order to achieve a desired response (e.g., a biological and/or therapeutic response) (e.g., the selectivity of the signal is established by the fact that it can be chosen to achieve the desired response). In a related embodiment of the invention, a device is provided for employing the methods of the invention described herein to apply electric and/or electromagnetic fields, generated by specific and selective signals, to up-regulate and/or down-regulate expression(s) of targeted gene(s).

In another embodiment of the invention, a method is provided for determining the specific and selective signal that regulates expression of a particular gene, such method preferably including (1) methodically varying (e.g., preferably by sequential dose-response curves) a first characteristic (e.g., duration, amplitude, duty cycle, frequency, waveform, and/or other characteristic) of a starting signal known to increase or suspected to increase cellular production of a given protein until a desired (e.g., preferably optimal) setting of the first characteristic is determined (based on measured amount of gene expression); (2) methodically varying a second characteristic of the signal (preferably in the same dose-response manner as with the first characteristic) while maintaining the first characteristic at the determined desired setting of the first characteristic, until a desired (e.g., preferably optimal) setting of the second characteristic is determined (based on measured amount of gene expression); (3) optionally methodically varying additional characteristics of the signal (preferably in the same dose-response manner as with the other characteristics), one at a time, while maintaining at their determined desired settings the characteristics for which desired settings have been determined, until desired (e.g., preferably optimal) settings of each of the additional characteristics are determined (based on measured amount of gene expression). It should be understood that each of the determined settings can be reviewed and/or adjusted at the end of the process, or during the process, to ensure their desired nature is maintained or established. It should be further understood that the characteristics need not be addressed in any particular order to achieve the present invention, but rather the total number of characteristics addressed can be adjusted, the type of characteristics can be different than those described, and the order in which the chosen characteristics are addressed can be changed, without departing from the scope of the invention. It should be further understood that one or more characteristics can be methodically varied simultaneously, rather than only one characteristic being methodically varied at a time.

In a preferred exemplary embodiment of this aspect of the invention, a method for determining a specific and selective signal that regulates expression of a particular gene preferably includes (1) methodically varying (e.g., preferably by performing sequential dose-response curves) an amplitude of a starting signal known to increase or suspected to increase cellular production of a given protein until an desired amplitude is determined (e.g., the amplitude corresponding to the maximum amount of gene expression observed), (2) methodically varying (preferably in the same dose-response manner as with the amplitude) the duration of the signal for at the determined amplitude until an desired duration is determined (e.g., the duration of time, applied at the determined amplitude, corresponding to the maximum amount of gene expression observed), (3) methodically varying (preferably in the same dose-response manner as with duration and with amplitude) the frequency of the signal for the determined duration of time at the determined amplitude until an desired frequency is determined (e.g., the frequency, applied at the determined amplitude and for the determined duration of time, corresponding to the maximum amount of gene expression observed); (4) methodically varying (preferably in the same dose-response manner as with duration and with amplitude and with frequency) the duty cycle of the signal for the determined duration of time at the determined amplitude and for the determined frequency until an desired duty cycle is determined (e.g., the duty cycle, applied at the amplitude and for the determined duration of time and at the determined frequency, corresponding to the maximum amount of gene expression observed); (5) methodically varying (preferably in the same dose-response manner as with duration and with amplitude and with duty cycle and with frequency) the waveform of the signal for the determined duration of time at the determined amplitude and for the determined duty cycle and at the determined frequency until a desired waveform is determined (e.g., the waveform, applied at the amplitude and for the determined duration of time and at the determined frequency and for the determined duty cycle, corresponding to the maximum amount of gene expression observed).

In another exemplary embodiment of this aspect of the invention, a method for determining a specific and selective signal that regulates VEGF expression preferably includes (1) methodically varying (e.g., preferably by performing sequential dose-response curves) an amplitude of a starting signal known to increase or suspected to increase cellular production of VEGF until desired amplitude is determined (e.g., the amplitude corresponding to a significant, even maximum, amount of VEGF expression observed), (2) methodically varying (preferably in the same dose-response manner as with amplitude) the duration of the signal at the determined amplitude until a desired duration is determined (e.g., the duration of time, applied at the determined amplitude, corresponding to the maximum amount of VEGF expression observed), (3) methodically varying (preferably in the same dose-response manner as with duration and with amplitude) the frequency of the signal for the determined duration of time at the determined amplitude until a desired frequency is determined (e.g., the frequency, applied at the amplitude and for the determined duration of time, corresponding to the maximum amount of VEGF expression observed); (4) methodically varying (preferably in the same dose-response manner as with duration and with amplitude and with frequency) the duty cycle of the signal for the determined duration of time at the determined amplitude and at the determined frequency until a desired duty cycle is determined (e.g., the duty cycle, applied at the amplitude and for the determined duration of time and at the determined frequency, corresponding to the maximum amount of VEGF expression observed); (5) methodically varying (preferably in the same dose-response manner as with duration and with amplitude and with duty cycle and with frequency) the waveform of the signal for the determined duration of time at the determined amplitude and for the determined duty cycle and at the determined frequency until a desired waveform is determined (e.g., the waveform, applied at the amplitude and for the determined duration of time and for the determined duty cycle and at the determined frequency, corresponding to the maximum amount of VEGF expression observed). It should be understood that each of the determined settings can be reviewed and/or adjusted at the end of the process, or during the process, to ensure their desired (e.g., optimum) nature is maintained or established. It should be further understood that the characteristics need not be addressed in any particular order to achieve the present invention, but rather the total number of characteristics addressed can be adjusted, the type of characteristics can be different than those described, and the order in which the chosen characteristics are addressed can be changed, without departing from the scope of the invention. It should be further understood that one or more characteristics can be methodically varied simultaneously, rather than only one characteristic being methodically varied at a time.

In an embodiment of the invention, a method is provided for treating tissue and/or a disease and/or condition (e.g., for treating peripheral vascular disease, cardiovascular disease, macular degeneration, wound healing, tendon and ligament healing, rheumatoid arthritis, bone healing (e.g., fresh fractures, fractures at risk, delayed healing and nonunion, bone defects, spine fusion, and as an adjunct in any of the above), and/or osteonecrosis, and/or for preventing tumor growth and spread), such method including (1) providing electric and/or electromagnetic fields that regulate VEGF expression in targeted tissue, which fields are generated by specific and selective signals suitable for generating such fields, and (2) exposing such targeted tissue to such fields so as to regulate VEGF expression therein. VEGF is a target gene of choice because, among other reasons, it is an important growth factor, and possibly the most important growth factor, in promoting vasculogenesis (in situ endothelial cell differentiation and proliferation to form new vessels) and angiogenesis (vessel sprouting or budding from pre-existing vessels).

In an exemplary embodiment of this aspect of the present invention, a method is provided for up-regulating VEGF expression in endothelial cells, such method preferably including (1) providing electric and/or electromagnetic fields that up-regulate VEGF expression in endothelial cells, which fields are generated by specific and selective signals suitable for generating such fields in endothelial cells, and (2) exposing endothelial cells to such fields (preferably via electrodes) so as to up-regulate VEGF expression in the endothelial cells. A desired (e.g., preferably effective for, and more preferably optimal for, generating an electric and/or electromagnetic field that up-regulates VEGF expression in endothelial cells) specific and selective signal is determinable by applying a method of the invention described above to perform sequential dose-response curves on chosen characteristics of a signal (e.g., duration, amplitude, frequency, and duty cycle), by which curves the effects of the resultant electric and/or electromagnetic field are measured. The signal presently determined to be most effective at generating a field that most effectively up-regulates VEGF expression in endothelial cells generates a capacitively coupled electric field with an amplitude of between 1 and 80 mV/cm inclusively, a duration of between 30 minutes and 24 hours inclusively, a frequency of between 30 and 120 kHz inclusively, and a duty cycle of between 5 and 100% inclusively, with a sine wave waveform. In particular, the present invention relates to up-regulating VEGF gene expression in endothelial cells via the application of fields generated by such signals. This method is useful for treating, among other diseases or conditions, peripheral vascular disease, cardiovascular disease, macular degeneration, wound healing, tendon and ligament healing, rheumatoid arthritis, bone healing (e.g., fresh fractures, fractures at risk, delayed unions, nonunion fractures, bone defects, spine fusion, and as an adjunct in any of the above), and/or osteonecrosis, and for preventing tumor growth and/or spread.

### BRIEF DESCRIPTION OF THE DRAWINGS

These and other aspects of the present invention will be elucidated in the accompanying drawings and following detailed description of the invention.

FIG. 1 is a graphic representation of aggrecan mRNA production by articular cartilage chondrocytes stimulated with a 20 mV/cm capacitively coupled electric field for various time durations. In this example, the response is time duration specific.

FIG. 2 is a graphic representation of the duration and magnitude of aggrecan mRNA up-regulation in articular cartilage chondrocytes following 30 minutes stimulation with a 20 mV/cm capacitively coupled electric field.

FIG. 3 is a graphic representation of aggrecan mRNA production in articular cartilage chondrocytes stimulated by various capacitively coupled electric field amplitudes, all for 30 minutes duration. In this example, the response is electric field amplitude specific.

FIG. 4 is a graphic representation of aggrecan mRNA production in articular cartilage chondrocytes stimulated by 20 mV/cm capacitively coupled electric field using various duty cycles. In this example, the response is duty cycle specific, and the duty cycle is time-wise selective.

FIG. 5 is a graphic representation of Type II collagen mRNA production in articular cartilage chondrocytes stimulated by a 20 mV/cm capacitively coupled electric field for various time durations. In this example, the response is time duration specific, similar to that of the complimentary aggrecan mRNA.

FIG. 6 is a graphic representation of the duration and magnitude of Type II collagen mRNA up-regulation in articular cartilage chondrocytes following 30 minutes stimulation with a 20 mV/cm capacitively coupled electric field.

FIG. 7 is a graphic representation of Type II collagen mRNA production in articular cartilage chondrocytes stimulated by various capacitively coupled electric field amplitudes, all for 30 minutes duration. This example shows that the differences between the field amplitude specificity of aggrecan mRNA (FIG. 3) and the amplitude specificity of Type II collagen mRNA allow for selectivity of signals.

FIG. 8 is a graphic representation of the down-regulation of MMP-1 mRNA production by articular cartilage chondrocytes treated with IL-β₁ and stimulated with a 20 mV/cm capacitively coupled field for various time durations. This example shows the selectivity and specificity of these electric fields whereby a specific signal must be used for a selected gene response.

FIG. 9 is a graphic representation of MMP-3 mRNA production by articular cartilage chondrocytes stimulated with a 20 mV/cm capacitively coupled electric field for various time durations. This example illustrates the significance of time specificity in the application of these signals.

FIG. 10 is a diagram illustrating a device for the treatment of osteoarthritis of the knee, in accordance with preferred embodiments of the present invention.

FIG. 11 is a diagram illustrating a nonunion of the femur stabilized by an intramedullary rod that is locked by two transcortical screws, and a device for the treatment of bone defects, in accordance with preferred embodiments of the present invention.

FIG. 12 is a diagram illustrating a device for the treatment of malignant melanoma, in accordance with preferred embodiments of the present invention.

FIG. 13 is a graphic representation of determinations regarding VEGF mRNA production by endothelial cells stimulated with 1, 20, 40, 60, and 80 mV/cm amplitude capacitively coupled electric fields. In this example, the response is amplitude specific.

FIG. 14 is a graphic representation of determinations regarding VEGF mRNA production by endothelial cells if stimulated with 15, 20, 25, 30, 35, and 40 mV/cm amplitude capacitively coupled electric fields. In this example, the response is amplitude specific.

FIG. 15 is a graphic representation of determinations regarding VEGF mRNA production by endothelial cells if stimulated with 20, 22.5, 30, 27.5, and 30 mV/cm amplitude capacitively coupled electric fields. In this example, the response is amplitude specific.

FIG. 16 is a graphic representation of determinations regarding VEGF mRNA production by endothelial cells stimulated with capacitively coupled electric field (of an amplitude desirable for VEGF mRNA production) for 0.5, 6, 12, 18, and 24 hour time periods. In this example, the response is time-duration specific.

FIG. 17 is a graphic representation of determinations regarding VEGF mRNA production by endothelial cells stimulated with capacitively coupled electric field (of an amplitude desirable for VEGF mRNA production) for 2, 4, 6, 8, and 10 hour time periods. In this example, the response is time-duration specific.

FIG. 18 is a graphic representation of determinations regarding VEGF mRNA production by endothelial cells stimulated with capacitively coupled electric field (of an amplitude desirable for VEGF mRNA production) for 5, 6.5, 6, 6.5, and 7 hour time periods. In this example, the response is time-duration specific.

FIG. 19 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production) at 30, 60, and 120 kHz frequencies. In this example, the response is frequency specific.

FIG. 20 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production) at 45, 60, and 75 kHz frequencies. In this example, the response is frequency specific.

FIG. 21 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production) at XX, XX, and XX kHz frequencies. In this example, the response is frequency specific.

FIG. 22 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production, at a frequency that has been determined to be desirable for VEGF mRNA production) at 5, 25, 50, 75, and 100% duty cycles. In this example, the response is duty-cycle specific.

FIG. 23 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production, at a frequency that has been determined to be desirable for VEGF mRNA production) at 40, 50, 60, and 70% duty cycles. In this example, the response is duty-cycle specific.

FIG. 24 is a graphic representation of determinations regarding VEGF mRNA production by endothelial cells stimulated with capacitively coupled electric field (of an amplitude that has been determined to be desirable for VEGF mRNA production, for a duration that has been determined to be desirable for VEGF mRNA production, at a frequency that has been determined to be desirable for VEGF mRNA production) at 40, 45, 50, 55, and 60% duty cycles. In this example, the response is duty-cycle specific.

FIG. 25 is a graphic representation of determinations regarding VEGF mRNA production by endothelial cells stimulated with capacitively coupled electric field (of an amplitude desirable for VEGF mRNA production) for 1, 2, 3, 4, 5, 6, and 24 hour time periods.

FIG. 26 is a graphic representation of determinations regarding VEGF mRNA production by endothelial cells if stimulated with 5, 10, 20, 30, 40 and 60 mV/cm amplitude capacitively coupled electric fields.

FIG. 27 is a diagram illustrating a device for the treatment of peripheral vascular disease, in accordance with preferred embodiments of the present invention.

### DETAILED DESCRIPTION OF EMBODIMENTS OF THE INVENTION

The invention will be described in detail below. Those skilled in the art will appreciate that the description given herein is for exemplary purposes only and is not intended in any way to limit the scope of the invention.

The present invention is based on the determination that the expression of certain genes can be regulated by applying electric and/or electromagnetic fields generated by specific and selective signals. In other words, it has been determined that there is a specific signal that generates an electric and/or electromagnetic field for regulating each gene in tissue (e.g., capillaries and blood vessels, the retina, healing wounds, tendons, ligaments, bone, cartilage, tumor cells, and other tissue cells), and that these specific signals are capable of regulating particular genes in such tissue. In particular, gene expression governing the growth, maintenance, repair, and degeneration or deterioration of tissue can be regulated in accordance with the invention by applying electric and/or electromagnetic fields generated by specific and selective signals so as to produce a salutory clinical effect. Such determinations are useful in the development of treatments for certain medical diseases and/or conditions (including but not limited to peripheral vascular disease, cardiovascular disease, macular degeneration, wound healing, tendon and ligament healing, rheumatoid arthritis, bone healing (e.g., fractures, fresh fractures, fractures at risk, delayed union, nonunion, bone defects, spine fusion), osteonecrosis, osteoarthritis, osteoporosis, and/or cancer, and for preventing tumor growth and spread, and as an adjunct in the treatment of any one or more of the above), as well as in the development of devices employing such methods.

As used herein, the term “signal” refers to any signal, including but not limited to mechanical signals, ultrasound signals, electromagnetic signals and electric signals.

As used herein, the term “field” refers to an electric and/or electromagnetic field within targeted tissue, regardless of type or method of generation. Examples include but are not limited to combined field, pulsed electromagnetic field, or generated by direct current, capacitive coupling or inductive coupling.

As used herein, the term “remote” is used to mean acting, acted on or controlled from a distance. As used herein, the phrase “remote regulation” refers to controlling from a distance (e.g., to “remotely regulate gene expression” refers to regulating the expression of a gene from a distance). As used herein, the phrase “to provide remotely” refers to providing from a distance. For example, providing a specific and selective signal from a remote source can refer to providing the signal from a source at a distance from a tissue or a cell, or from a source outside of or external to the body.

As used herein, the term “regulate” means to control gene expression, and is understood to include both up-regulate and down-regulate. As used herein, the term “up-regulate” means to increase expression of a gene, and the term “down-regulate” means to inhibit or prevent expression of a gene.

As used herein, the phrase “functionally complementary” refers to two or more genes whose expressions are complementary or synergistic in a given cell or tissue.

As used herein, the term “tissue” refers to an aggregate of cells together with their extracellular substances that form one of the structural or other materials of a patient. As used herein, the term “tissue” is intended to include any tissue of the body including but not limited to capillaries, blood vessels, muscle and organ tissue, wound tissue, tumor tissue, bone tissue, or cartilage tissue. Also, the term “tissue” as used herein may also refer to an individual cell.

As used herein, the term “patient” refers to an animal, preferably a mammal, more preferably a human.

The present invention provides treatment methods and devices that target certain tissues and/or diseases and/or conditions. In particular, gene expression associated with the repair process in tissue can be regulated by the application of signals, and electric and/or electromagnetic fields generated by such signals that are effective for regulating gene expression in the target tissue. Gene expression can be up-regulated or down-regulated by the application of electric and/or electromagnetic fields generated by signals that are specific and selective for regulating expression of each gene or each set of functionally complementary genes so as to produce a desired (e.g., preferably beneficial) clinical effect. For example, an electric and/or electromagnetic field generated by a particular specific and selective signal may up-regulate a certain desirable gene expression, while the same or another field generated by a particular specific and selective signal may down-regulate a certain undesirable gene expression. A certain gene expression may be up-regulated by an electric and/or electromagnetic field generated by one particular specific and selective signal and down-regulated by an electric and/or electromagnetic field generated by another specific and selective signal. Those skilled in the art will understand that certain tissues and/or diseases and/or conditions can be treated by regulating expression of those genes governing the growth, maintenance, repair, and degeneration or deterioration of the implicated tissues.

The methods and devices of the present invention are based on identifying those signals that are specific and selective for generating electric and/or electromagnetic fields that regulate the gene expression associated with treating certain tissue, diseases, and/or conditions. For example, electricity in its various forms (e.g., capacitive coupling, inductive coupling, combined fields) can specifically and selectively regulate gene expression in tissue by varying one or more characteristics (e.g., frequency, amplitude, waveform, or duty cycle) of the signal generating the applied electric and/or electromagnetic field for the gene of interest. Other characteristics (e.g., the duration of time applied) of the signal can also influence the capability of the field to regulate gene expression in targeted tissue. Specific and selective signals may generate fields for application to each gene systematically or otherwise until the proper combination of characteristics (e.g., frequency, amplitude, waveform, duty cycle, and duration) is found that provides the desired effect on gene expression.

It is to be understood that a variety of diseased or injured tissues, conditions, or disease states can be targeted for treatment because the specificity and selectivity of a signal that generates an electric and/or electromagnetic field to regulate expression of a certain gene can be influenced by several factors. In particular, for example, a signal of appropriate frequency, amplitude, waveform, and/or duty cycle can be specific and selective for generating an electric and/or electromagnetic field that regulates the expression of certain genes and thus provide for targeted treatments. Temporal factors (e.g., duration of time exposed to the field) can also influence the specificity and selectivity of a signal that generates an electric and/or electromagnetic electric field for regulating expression of a particular gene. That is, the regulation of gene expression may be more effective (or made possible) by applying an electric and/or electromagnetic field for a particular duration of time. Therefore, those skilled in the art will understand that the present invention provides for varying the frequency, amplitude, waveform, duty cycle, and/or duration of application of a signal that generates an electric and/or electromagnetic field until the field is found to regulate certain gene expressions more effectively in order to provide for treatments targeting a variety of diseased or injured tissue or diseases.

Thus, the present invention provides for targeted treatments because it is possible to regulate expression of certain genes associated with a particular diseased or injured tissue, or a particular disease or condition, via the application of electric and/or electromagnetic fields generated by specific and selective signals of appropriate frequency, amplitude, waveform and/or duty cycle for an appropriate duration of time. The specificity and selectivity of a signal generating an electric and/or electromagnetic field may thus be influenced so as to regulate the expression of certain genes in order to target certain diseased or injured tissue, conditions, or disease states for treatment. The present invention thereby provides for a multitude of targeted treatments including the treatment of osteoarthritis, osteoporosis, cancer, peripheral vascular disease, cardiovascular disease, macular degeneration, wound healing, tendon and ligament healing, rheumatoid arthritis, bone healing (e.g., fresh fractures, fractures at risk, delayed healing, nonunion, bone defects, spine fusion, and as an adjunct to any of the above) osteonecrosis, and/or as an adjunct in the treatment of one or any of the above, and in preventing tumor growth and spread.

The present invention further provides devices for the treatment of injured or diseased tissue, conditions, and disease states. In particular, the present invention provides devices that include a source of at least one signal that is specific and selective for generating an electric and/or electromagnetic field that regulates expression of a gene. The devices of the present invention can provide for the production of such signals or fields for application to the targeted tissue by at least one electrode adapted to apply the specific and selective signal.

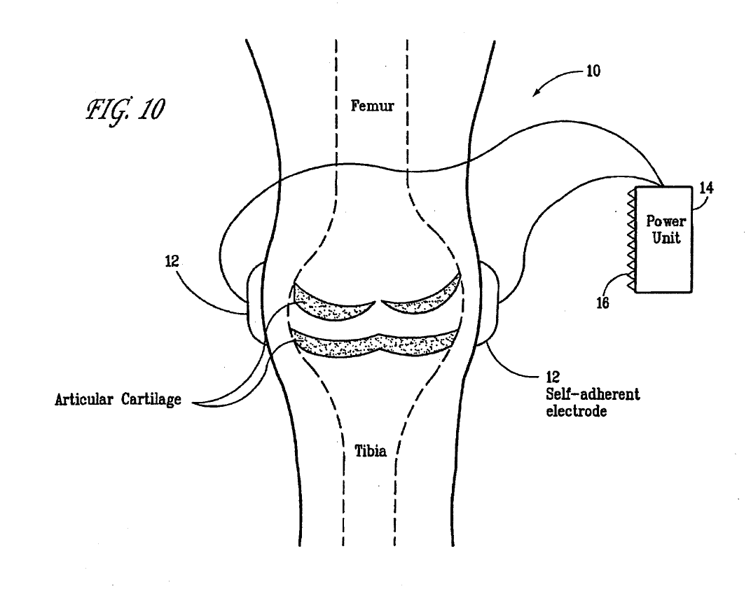

The devices of the present invention are capable of applying specific and selective signals, and consequently an electric and/or electromagnetic field generated by specific and selective signals, directly to diseased or injured tissue and/or indirectly to diseased or injured tissue (e.g., the signal can be applied to the skin of a patient near the targeted tissue). The devices of the present invention may also provide for the remote application of specific and selective signals, or an electric and/or electromagnetic field generated by specific and selective signals (e.g., remote application being application of a signal, or an electric and/or electromagnetic field generated by a signal, at a distance from targeted tissue (e.g, diseased or injured tissue) yet which yields the desired effect on the patient's body and/or within the targeted tissue), although it will be appreciated that capacitively coupled devices must touch the patient's skin. The devices of the present invention may include means for attaching electrodes to the body of a patient in the vicinity of injured or diseased tissue, such as in the case of capacitive coupling. For example, self-adherent conductive electrodes may be attached to the skin of the patient on both sides of a fractured bone, or both sides of a knee joint afflicted with osteoporosis as shown in FIG. 10. As also shown in FIG. 10, the devices of the present invention may alternatively or additionally include means, such as, for example, self-adherent electrodes, for attaching the device to the body of a patient. For example, the devices of the present invention may include electrodes attached to a power unit that has a VELCRO® patch on the reverse side such that the power unit can be attached to a VELCRO® strap (not shown) fitted around (e.g., around the calf, thigh or waist of, or a cast on) the patient. In the case of inductive coupling, the device of the present invention may include coils attached to a power unit in place of electrodes.

The devices of the present invention can be employed in a variety of ways. The devices may be portable or may be temporarily or permanently attached to a patient's body. The devices of the present invention are preferably non-invasive. For example, the devices of the present invention may be applied to the skin of a patient by application of electrodes adapted for contact with the skin of a patient for the application of predetermined specific and selective signals, or electric and/or electromagnetic fields generated by predetermined specific and selective signals. Such signals may also be applied via coils in which time varying currents flow, thus producing electric and/or electromagnetic fields that penetrate the tissue and create the electric and/or electromagnetic fields in the targeted tissue. The devices of the present invention may also be capable of implantation in a patient, including implantation under the skin of a patient.

Examples below will illustrate that the methods of the present invention may provide for bone growth and repair via regulation of gene expression in bone cells. The methods of the present invention can stimulate bone growth and repair in the vicinity of fresh fractures and non-union fractures. Bone growth and repair also can be stimulated in the vicinity of osteoarthritis or osteoporosis. A variety of cells can be targeted by the methods of the present invention including capillary and blood vessel cells, muscle cells, organ cells, bone cells, cartilage cells, retinal cells, tendon cells, ligament cells, fibrous tissue cells, stem cells, tumor cells, cancer cells, and other tissue.

Examples below also will illustrate that the methods of the present invention may provide for cartilage growth and repair. Cartilage growth and repair can be stimulated by applying electric and/or electromagnetic fields generated by signals that are specific and selective for the expression of certain genes. For example, the methods of the present invention can stimulate articular cartilage repair in osteoarthritis patients and provide for the regulation of gene expression in cartilage cells. In particular, the methods of the present invention can provide for the up-regulation of genes that repair cartilage (e.g., genes encoding for aggrecan and Type II collagen), down-regulation of genes that destroy cartilage (e.g., genes encoding for metalloproteinase) and the up-regulation of genes that inhibit metalloproteinases that destroy articular cartilage (e.g., genes encoding for tissue inhibitors of metalloproteinase). A variety of cartilage cells can be targeted by the methods of the present invention including articular chondrocytes and including articular cartilage, hyaline cartilage, and growth plate cartilage.

Examples below further illustrate that the methods of the present invention provide for the regulation of gene expression in articular chondrocytes. For example, in the examples below, fetal articular chondrocytes have been exposed to a capacitively coupled 60 kHz electrical field of 20 mV/cm for 0.5, 2.0, 6.0 and 24.0 hours. A statistically significant incorporation of ³⁵SO₄/ug DNA (indicating significant proteoglycan synthesis) was found after only 0.5 hours of stimulation. An identical experiment was repeated and the levels of aggrecan mRNA, the messenger for the major cartilage proteoglycan, monitored. After only 0.5 hours of electrical stimulation there was a significant increase (almost 100%) in aggrecan mRNA. Accordingly, temporal factors may influence the specificity and selectivity of a signal that generates an electric and/or electromagnetic field regulating gene expression in articular chondrocytes.

Examples below further illustrate that the methods of the present invention provide for the regulation of VEGF in endothelial cells. By performing sequential dose-response curves on characteristics (e.g., duration, amplitude, frequency, duty cycle, and/or waveform) of a signal, by which curves the effects of the electric and/or electromagnetic field generated by the signal are measurable, a desired (e.g., preferably optimal) signal for up-regulating VEGF mRNA in endothelial cells is determinable. The signal presently determined to be most effective at generating a field that most effectively up-regulates VEGF expression in endothelial cells generates a capacitively coupled electric field with an amplitude of between 1 and 80 mV/cm inclusively, a duration of between 30 minutes and 24 hours inclusively, a frequency of between 30 and 120 kHz inclusively, and a duty cycle of between 5 and 100% inclusively, with a sine wave waveform. In particular, the present invention relates to up-regulating VEGF gene expression in endothelial cells via the application of fields generated by such signals. Increasing VEGF gene expression in endothelial cells is a viable treatment for peripheral vascular disease because vasculogenesis and/or angiogenesis are increased to bring an increased blood supply to the ischemic limb.

The methods of the present invention also provide for the treatment of certain diseases. In particular, the methods of the present invention can provide for the treatment of cancer. In a patient with a primary (or even metastatic) cancer, metalloproteinase is at least partly responsible for spread of the cancer. Metalloproteinase enzymatically breaks down fibrous walls or membranes erected by adjacent cells in an attempt to contain the cancer. However, as mentioned above, tissue inhibitors of metalloproteinase may inhibit the production of such metalloproteinases. Accordingly, methods of the present invention can provide for the down-regulation of genes encoding for metalloproteinase and the up-regulation of genes encoding for tissue inhibitors of metalloproteinase (“TIMP”). Those skilled in the art will understand that a variety of other diseases and conditions may be targeted for treatment via the methods of the present invention, such as peripheral vascular disease, cardiovascular disease, macular degeneration, wound healing, tendon and ligament healing, rheumatoid arthritis, bone healing (e.g., fresh fractures, fractures at risk, delayed healing and nonunion, bone defects, spine fusion, and as an adjunct in any of the above), osteonecrosis, tumor growth and spread, and/or other diseases or conditions.

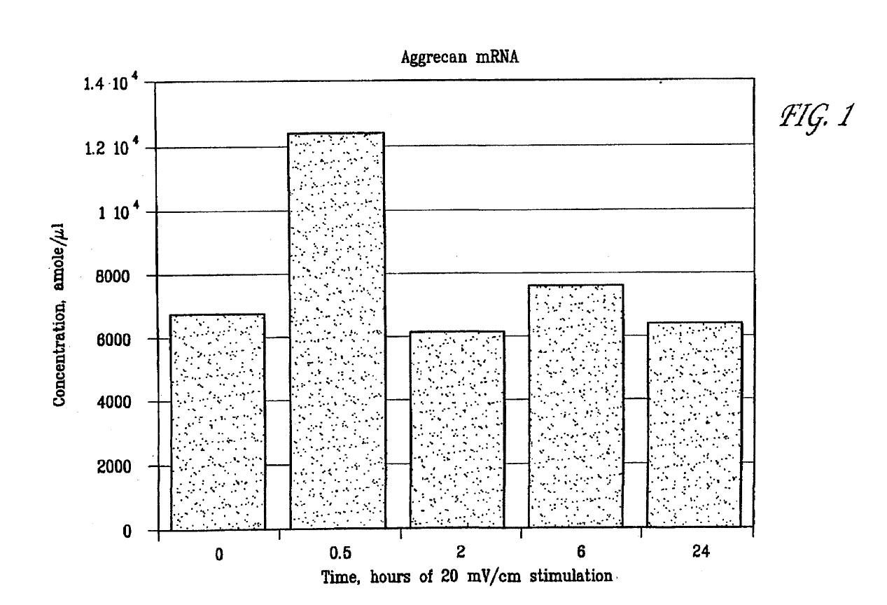

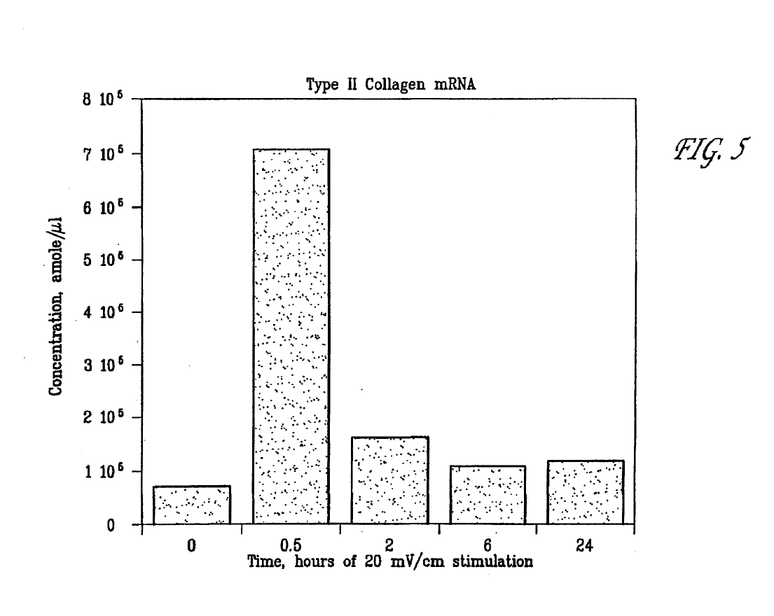

While not limiting the present invention in any way, it is presently believed that those genes that are functionally complementary may respond to identical or substantially similar signals. In other words, a signal may be specific and selective for generating an electric and/or electromagnetic field that regulates expression of functionally complementary genes. With reference to FIGS. 1 and 5, and as described below with respect to Examples 1 and 2, those genes encoding aggrecan and Type II collagen can both be regulated by a 20 mV/cm, 60 kHz capacitively coupled signal. Each of these genes regulates cartilage matrix formation and is thus believed to be functionally complementary. On the other hand, as described below with respect to Example 5, a 20 mV/cm, 60 kHz capacitively coupled signal regulates the gene expression for encoding TGF-β₁ but does not regulate the gene expression for PDGF-A. Each of these genes participates in the regulation of different phases and physiologic processes of bone healing and are thus are not believed to be functionally complementary.

FIGS. 10-12 and 25 provide example of the devices of the present invention. Devices of the present invention can include a source of specific and selective signals, a power unit, and at least one electrode. Devices of the present invention can be portable. For example, the electrodes may be attached to a power unit that can be attached to a VELCRO® strap which can be fitted around a targeted body area (such as, for example, the calf, thigh, or waist) of a patient. Such a device can be used to apply an electric and/or electromagnetic field generated by a specific and selective signal for a particular duration (e.g., 30 minutes or more per day) so as to regulate expression of a particular gene (e.g., aggrecan, Type II collagen, or VEGF).

Those skilled in the art will further understand that the devices of the present invention can be provided in a variety of forms including a capacitively coupled power unit with programmed, multiple, switchable, specific and selective signals for application to one pair or to multiple pairs of electrodes, electromagnetic coils, or a solenoid attached to a power unit with switchable, multiple, specific and selective signals, and an ultrasound stimulator with a power supply for generating specific and selective signals. Generally speaking, device preference is based on patient acceptance and patient compliance. The smallest and most portable unit available in the art at the present time is a capacitive coupling unit; however, patients with extremely sensitive skin may prefer to use inductive coupling units. On the other hand, ultrasound units may be used, and require the most patient cooperation, but may be desirable for use by certain patients.

### EXAMPLES

The invention is further demonstrated in the following examples, which are for purposes of illustration, and are not intended to limit the scope of the present invention.

Chondrocyte cultures were prepared from fetal bovine articular cartilage. Chondrocytes (5×10⁵ cells/cm²) were plated onto specially modified Cooper dishes. The cells were grown to seven days with the medium changed just prior to beginning the experimental condition. The experimental cell cultures throughout these studies were subjected to a capacitively coupled 60 kHz sine wave signal electric field with an output of 44.81 volts peak to peak. This produced a calculated-field strength in the culture medium in the dishes of 20 mV/cm with a current density of 300 μA/cm². Control cell culture dishes were identical to that of the stimulated dishes except that the electrodes were not connected to a function generator.

Total RNA was isolated using TRIzol, according to the manufacturer's instructions, and reversed transcription using SuperScript II reverse transcriptase was performed. Oligonucleotide primers to be used in the competitive PCR technique were selected from published cDNA sequences. Quantitative analysis of PCR products was performed using ScionImage software.

A signal with desired characteristics for generating a field to effect the desired gene regulation was determined as follows. A signal known to increase (or suspected to increase) cellular production of a given protein is taken as the starting signal for determining the specific signal for generating a field to regulate the gene expression (mRNA) of that protein. A dose-response curve is first performed by varying the duration of the signal while holding other chosen signal characteristics constant (e.g., amplitude, duty-cycle, frequency, and waveform). This determines a desired (and potentially optimal) duration of the starting signal for generating a field to regulate the gene expression of that protein. A second dose-response curve is performed by varying the amplitude for a desired duration of time. This determines a desired amplitude for the desired duration of time as determined by the gene expression of the protein of interest. A third dose-response curve is then performed, this time varying the duty-cycle from 100% (constant) to 1% or less while holding the desired amplitude and other chosen signal characteristics constant. A dose-response curve is repeated a fourth time (varying frequency) and a fifth time (varying waveform) each time keeping the other chosen signal characteristics constant (preferably at optimal settings). By this method a desired, and preferably an optimal, signal is determined for generating a field that produces the greatest increase in the gene expression of the protein of interest.

Protein expression may be determined by any method known in the art, such as reverse transcriptase PCR, Northern analysis, immunoassays, and the like.

### Example 1

### Aggrecan Production by Articular Chondrocytes

Articular chondrocytes were exposed to a capacitively coupled electric signal of 20 mV/cm at 60 kHz. The results are illustrated in FIGS. 1-4.

FIG. 1 is a graphic representation of aggrecan mRNA production by articular cartilage chondrocytes (attomole per μl) stimulated with a 20 mV/cm capacitively coupled electric field for time durations of 0 (control), 0.5, 2, 6, and 24 hours. In this example, 30 minutes stimulation was found to provide a significant increase (almost a two-fold increase) in aggrecan mRNA. The response is thus time duration specific.

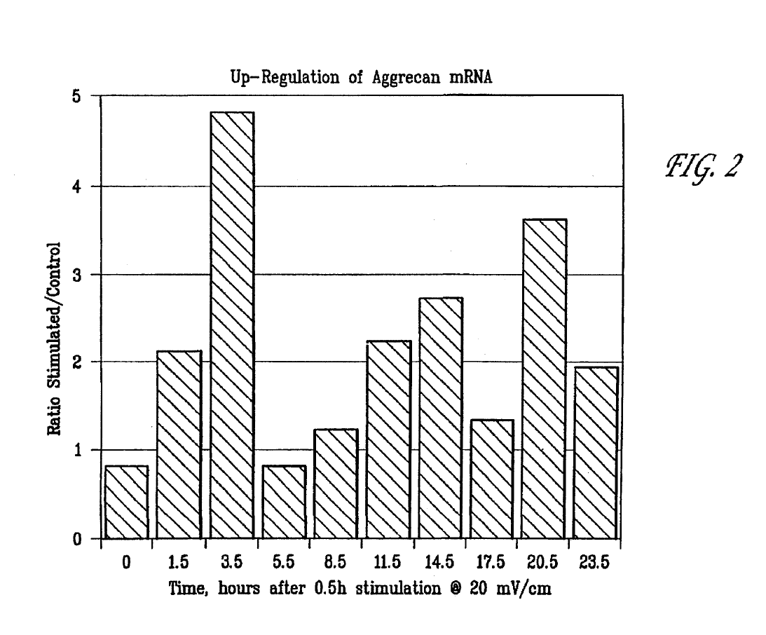

FIG. 2 is a graphic representation of the duration and magnitude of aggrecan mRNA up-regulation in articular cartilage chondrocytes following 30 minutes stimulation with a 20 mV/cm (60 kHz) capacitively coupled electric field. As illustrated, it was found that the peak up-regulation occurs 3½ hours following the cessation of the 30 minute stimulation period. FIG. 2 also illustrates that the up-regulation is cyclic, with secondary, smaller peaks of up-regulation occurring 14½ hours and 20½ hours after cessation of the 30 minute stimulation period.

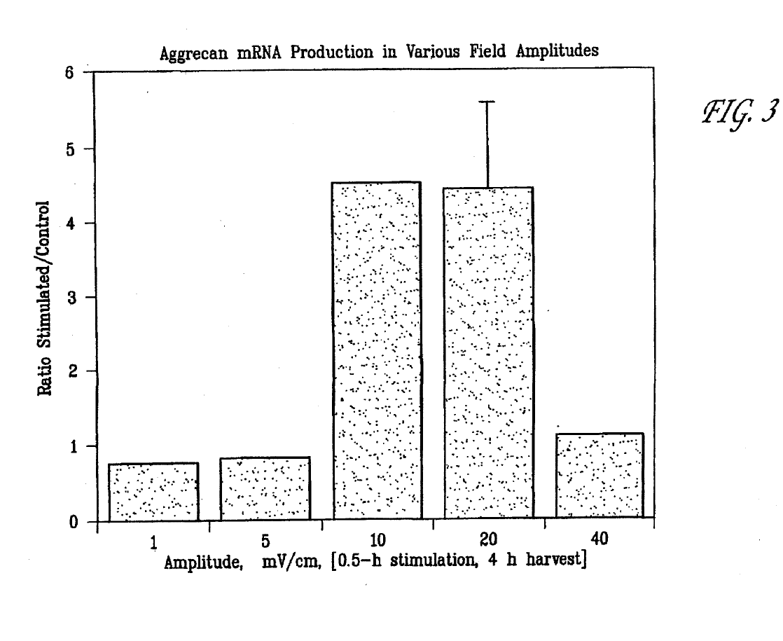

FIG. 3 is a graphic representation of aggrecan mRNA production in articular cartilage chondrocytes stimulated by various capacitively coupled electric field amplitudes, all for 30 minutes duration. In this example, 10-20 mV/cm showed significant increases in aggrecan mRNA production. Thus, the response is electric field amplitude specific.

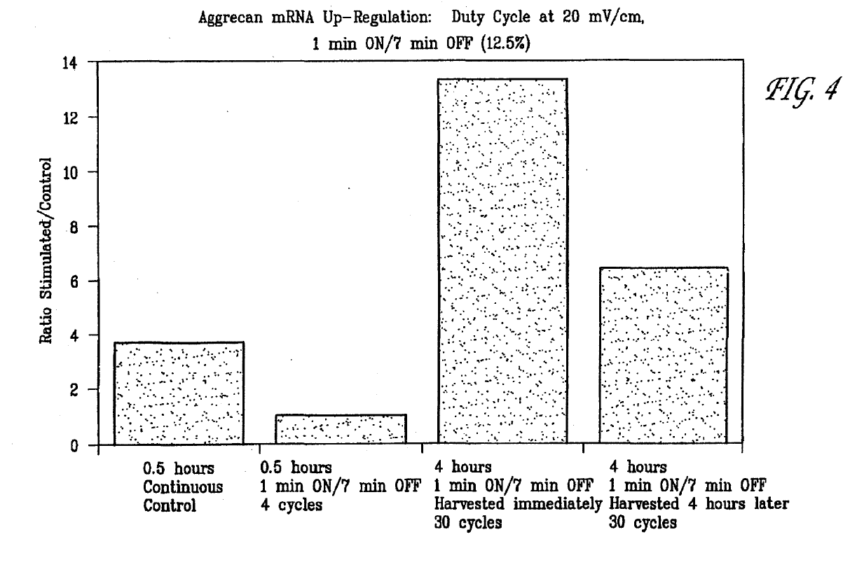

FIG. 4 is a graphic representation of aggrecan mRNA production in articular cartilage chondrocytes stimulated by 20 mV/cm (60 kHz) capacitively coupled electric field using various duty cycles. As illustrated, a duty cycle of 1 minute on/7 minutes off (12/5% duty cycle) pulsed for 30 cycles (total “on” time of stimulation=30 minutes) leads to a far greater production of aggrecan mRNA than 30 minutes of constant (control, 100% duty cycle) stimulation. The response is thus duty cycle specific. FIG. 4 also illustrates that a 1 minute on/7 minute off (12.5% duty cycle) signal for 4 hours gives significantly more aggrecan mRNA than does the same 12.5% duty cycle applied for 30 minutes. The duty cycle is thus time-wise selective.

### Example 2

### Type II Collagen Production by Articular Chondrocytes

Articular chondrocytes were exposed to a capacitively coupled electric signal of 20 mV/cm at 60 kHz. The results are illustrated in FIGS. 5-7.

FIG. 5 is a graphic representation of Type II collagen mRNA production (attomole per .mu.l) in articular chondrocytes stimulated by a 20 mV/cm (60 kHz) capacitively coupled electric field for time durations of 0 (control), 0.5, 2, 6 and 24 hours. In this example, 30 minutes of stimulation provided a significant increase (approximately ten-fold increase) in collagen Type II mRNA. This shows that the response is time duration specific, similar to that of the complementary aggrecan mRNA of Example 1.

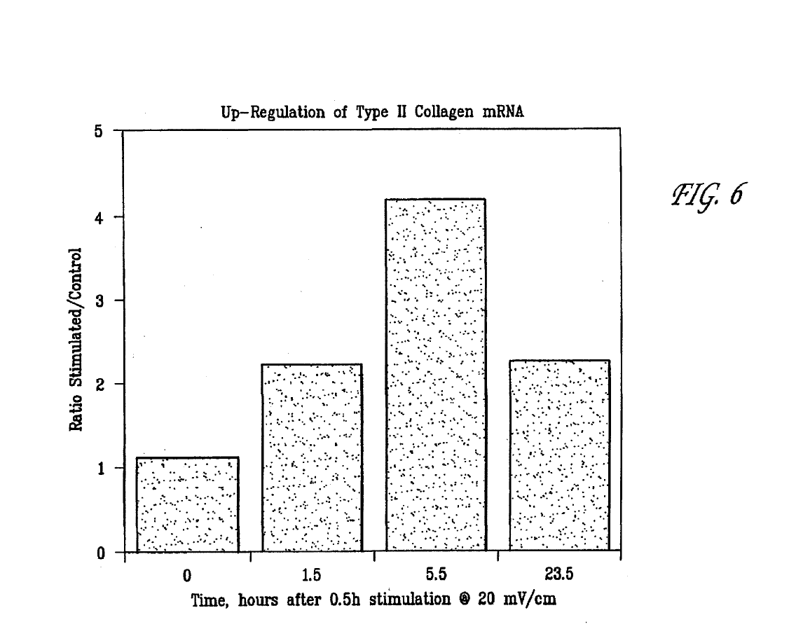

FIG. 6 is a graphic representation of the duration and magnitude of Type II collagen mRNA up-regulation in articular chondrocytes following 30 minutes stimulation with a 20 mV/cm capacitively coupled electric field. FIG. 6 illustrates that peak up-regulation occurs 5½ hours following cessation of the 30 minute stimulation period. It is noteworthy that aggrecan mRNA, a complementary gene, reached a maximum production of aggrecan mRNA at 3½ hours after cessation of stimulation, 2 hours earlier than with Type II collagen mRNA (FIG. 2).

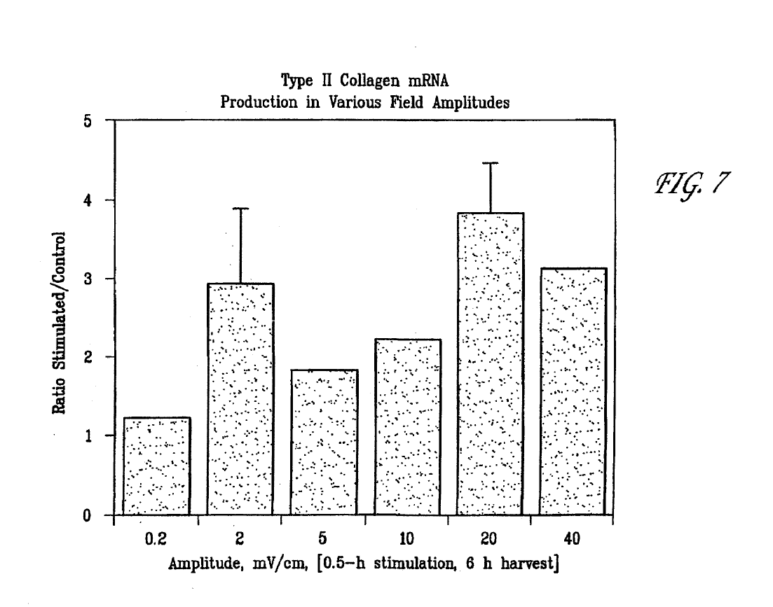

FIG. 7 is a graphic representation of Type II collagen mRNA production in articular chondrocytes amplitudes, all for 30 minutes duration. As illustrated, 20, 40, and 2 mV/cm all showed significant increases in Type II collagen mRNA. It is also noteworthy that the differences between the field amplitude specificity of aggrecan mRNA (FIG. 3) and the amplitude specificity of Type II collagen mRNA allow for selectivity of signals. For example, one could selectively choose a 10 mV/cm signal to stimulate aggrecan mRNA if one did not want to stimulate Type II collagen mRNA, or a 2 mV/cm or a 40 mV/cm signal to stimulate Type II collagen mRNA if one did not want to stimulate aggrecan mRNA. This data shows that the specificity of the applied signals allows one to obtain a specific gene expression.

With reference to Examples 1 and 2, it is demonstrated that each of those genes encoding aggrecan or Type II collagen can be regulated by an identical 20 mV/cm, 60 kHz capacitively coupled signal. Those skilled in the art will appreciate that each of these gene transcripts regulates cartilage matrix formation and are functionally complementary. Accordingly, the findings of Examples 1 and 2 are believed to support electrical therapy through gene regulation in accordance with the techniques described herein.

### Example 3

### MMP-1 mRNA Production in IL-β₁ Treated Articular Chondrocytes

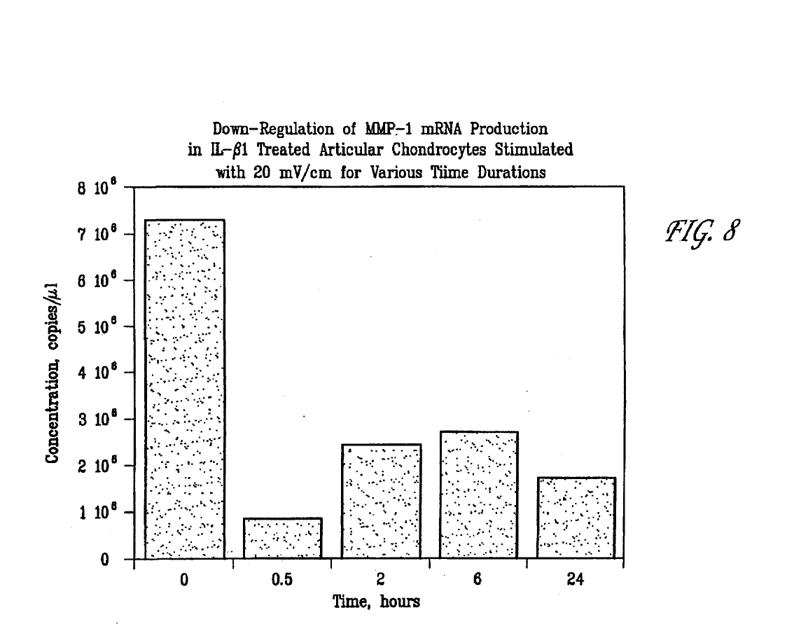

Articular chondrocytes were exposed to a capacitively coupled electric signal of 20 mV/cm at 60 kHz. The results are illustrated in FIG. 8.

FIG. 8 is a graphic representation of MMP-1 mRNA production by articular cartilage chondrocytes treated with IL-β₁ and stimulated with a 20 mV/cm (60 kHz) capacitively coupled field for time durations of 0 (control), 0.5, 2, 6, and 24 hours. As illustrated, MMP-1 mRNA is dramatically down-regulated in all time durations of stimulation, but especially so at 30 minutes. This is significant when contrasted with the dramatic up-regulation of aggrecan mRNA (FIGS. 1-4) and Type II collagen mRNA (FIGS. 5-7) in the same 20 mV/cm field. This shows the selectivity and specificity of these electric fields whereby a specific signal must be used for a selected gene response.

### Example 4

### MMP-3 mRNA Production in IL-β₁ Treated Articular Chondrocytes

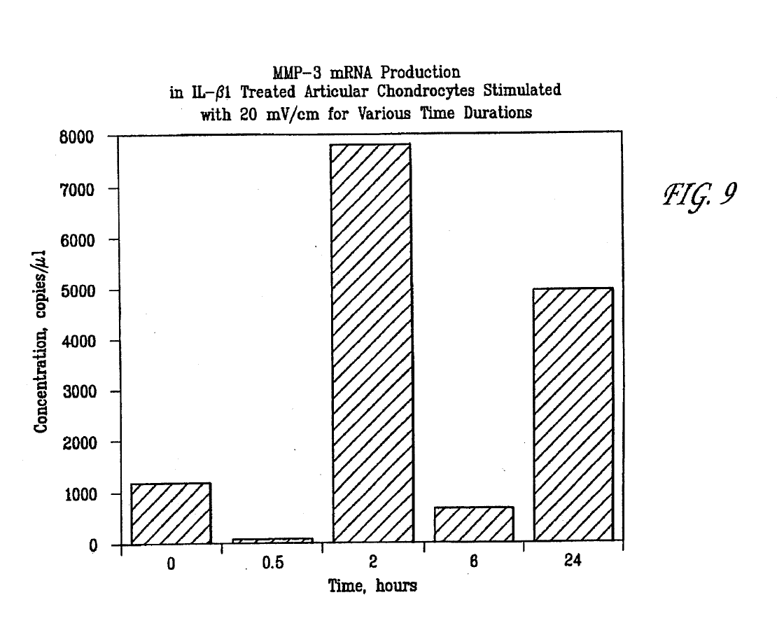

Articular chondrocytes were exposed to a capacitively coupled electric signal of 20 mV/cm at 60 kHz. The results are illustrated in FIG. 9.

FIG. 9 is a graphic representation of MMP-3 mRNA production by articular cartilage chondrocytes stimulated with a 20 mV/cm (60 kHz) capacitively coupled electric field for time durations of 0 (control), 0.5, 2, 6, and 24 hours. As illustrated, there is significant down-regulation of MMP-3 mRNA with 30 minutes of stimulation and a dramatic up-regulation with 2 hours of stimulation. This points out the significance of time specificity in the application of these signals.

### Example 5

### TGF-β₁ Production by Bone Cells

As noted above, it has been reported that a 60 kHz capacitively coupled electric field of 20 mV/cm produces a significant increase in TGF-β₁ in similar bone cells. Brighton et al., Biochem. Biophys. Res. Commun. 237: 225-229 (1997). It was found that there was significant production of TGF-β₁, mRNA, but only after 6 hours of stimulation (in contrast to 0.5 hours for aggrecan mRNA and Type II collagen mRNA). The experiment was repeated to determine if the exposure of MC3T3-E1 bone cells to the 20 mV/cm, 60 kHz capacitively coupled electric signal had an effect on the production of PDGF-A mRNA. No effect was found.

Thus, a 20 mV/cm, 60 kHz capacitively coupled signal regulates bone cell genes encoding TGF-β₁ but fails to regulate genes encoding PDGF-A. It is presently understood that the expression of each of these genes participates in the regulation of different phases and physiologic processes of bone healing and are thus are not functionally complementary.

### Example 6

### Treatment of Osteoarthritis

With reference to FIG. 10, a device 10 in accordance with preferred embodiments of the present invention is used to treat a patient with osteoarthritis of the knee. As illustrated, two circular, soft conductive, self-adherent electrodes 12 are placed on the skin on either side of the knee at the level of the joint line. The electrodes 12 are attached to a power unit 14 which has a VELCRO® patch 16 on the reverse side such that the power unit 14 can be attached to a VELCRO® strap (not shown) fitted around the calf, thigh or waist. The electrodes 12 may be placed on the skin before the patient goes to bed each evening or any other time.

The power unit is preferably small (e.g., 6-8 ounces) and powered by a standard 9-volt battery to emit a 5 volt peak-to-peak, 6-10 mAmp, 20 mV/cm, 60 kHz sine wave signal to the electrodes 12 placed on the skin. As illustrated in the above examples, this signal provided 30 minutes per day with the desired time duration, field amplitude, and duty cycle should significantly up-regulate genes encoding aggrecan and Type II collagen. This treatment should prevent or minimize further articular cartilage deterioration as well as heal articular cartilage that already is damaged or degenerated.

The power unit 14 also may be reconfigured to provide signals specific and selective for other genes. For example, as illustrated in the above examples, the power unit 14 may be reconfigured to provide signals for down-regulating the gene expression of metalloproteinase (MMP) as well as signals for up-regulating genes expressing tissue inhibitors of metalloproteinase (“TIMP”) genes. The power unit 14 may be reconfigured to provide such signals in sequence with the aggrecan/Type II collagen signal. Accordingly, the patient may be treated through the up-regulation of genes that repair cartilage (e.g., aggrecan and Type II collagen genes), down-regulation of genes that destroy cartilage (e.g., metalloproteinase gene) and the up-regulation of genes that inhibit the metalloproteinases that destroy articular cartilage (e.g., tissue inhibitors of metalloproteinase).

### Example 7

### Treatment of Bone Defects or Osteoporosis

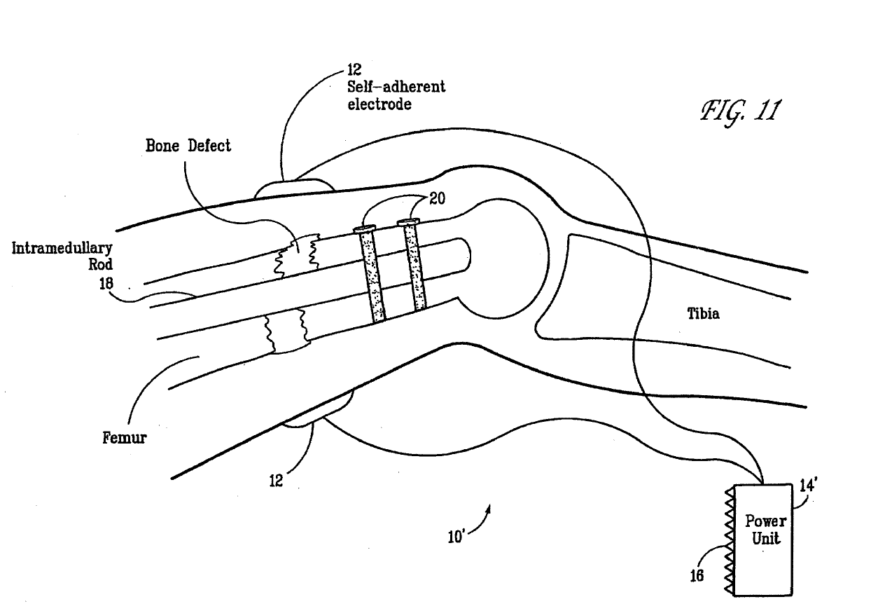

With reference to FIG. 11, a patient with a fracture, delayed union, nonunion or other bone defect may be treated with two circular, soft conductive electrodes 12 placed on the skin on opposite sides of the extremity at the level of the defect. The electrodes 12 are placed on the skin so as to span the bone defect. The electrodes 12 are attached to a power unit 14′ which has a VELCRO® patch 16 on the reverse side such that the power unit 14′ can be attached to a VELCRO® strap (not shown) fitted around the calf, thigh or waist. In accordance with preferred embodiments of the invention, a nonunion of the femur may be stabilized by an intramedullary rod 18 locked by two transcortical screws 20, as shown in FIG. 11.

The power unit 14′ provides a 20 mV/cm, 60 kHz sine wave signal to the electrodes 12 placed on the skin. The signal is provided for 6 hours per day as in example 5. The power unit 14′ is differentiated from power unit 14 in the previous example since the same electrical signal as defined by time duration, field amplitude, and duty cycle is not necessarily applied. This technique should aid in the repair process by up-regulating TGF-β₁, a gene important in the cartilage phase of bone repair.

Those skilled in the art will appreciate that the power unit 14′ may be reconfigured to provide other signals specific for certain genes. For example, the power unit 14 may be reconfigured to provide signals for the up-regulation of PDGF-A, basic FGF and BMP-2 genes. The power unit 14 also may be reconfigured to provide in sequence those signals specific and selective for TGF-β₁, PDGF-A, basic FGF, and BMP-2 genes. Therefore, the power unit 14 may be reconfigured to provide specific and selective signals that up-regulate genes necessary to heal bone defects.

### Example 8

### Treatment of Tumors

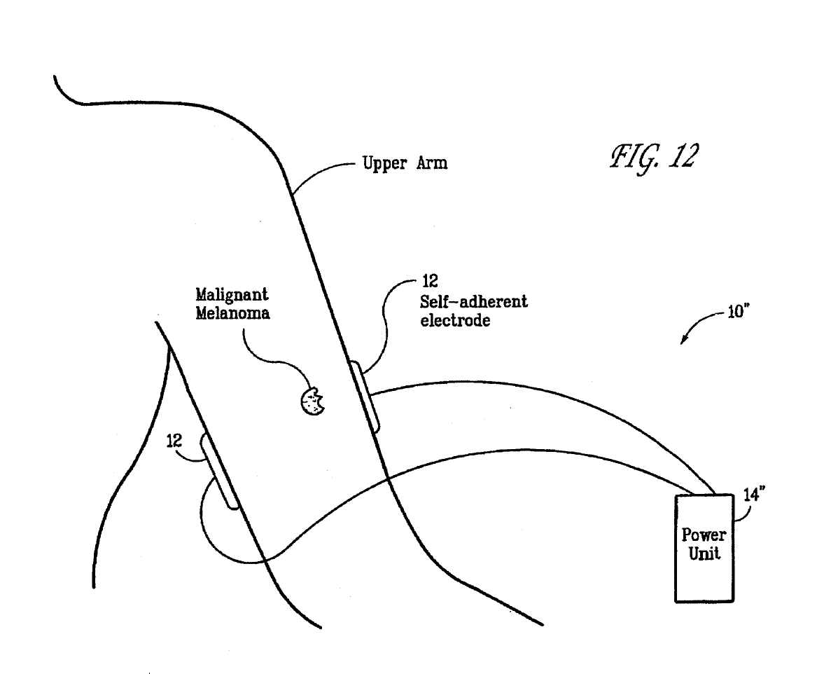

With reference to FIG. 12, a patient with malignant melanoma may be treated with methods and devices according to preferred embodiments of the present invention. FIG. 12 shows a patient with malignant melanoma that has not yet broken out of the skin into the underlying tissue. As discussed above, in a patient with a primary (or even metastatic) cancer, spread of the cancer takes place by metalloproteinases, which are produced by cancer cells. Metalloproteinases enzymatically break down the fibrous wall or membrane that adjacent cells establish in an attempt to contain the cancer. As discussed above, tissue inhibitors of metalloproteinase may inhibit the production of such metalloproteinases.

The device 10″ of the invention provides specific capacitively coupled electric fields via electrodes 12 for selectively down-regulating the gene encoding for metalloproteinase as discussed in the above examples and/or selectively up-regulating the gene encoding for tissue inhibitors of metalloproteinase (“TIMP”) The device 10″ can provide the electric field generated by power unit 14″ so as to selectively down-regulate and up-regulate the genes sequentially for specific periods of time per day. The melanoma can be safely excised once the melanoma has been sufficiently encapsulated by the body's own defensive mechanism.

### Example 9

### VEGF mRNA Production by Endothelial Cells

A signal determined to be desirable for generating a field that effectively up-regulates VEGF expression in endothelial cells as measured by mRNA production generates a capacitively coupled electric field with an amplitude of between 1 and 80 mV/cm inclusively, a duration of between 30 minutes and 24 hours inclusively, a frequency of between 30 and 120 kHz inclusively, and a duty cycle of between 5 and 100% inclusively, with a sine wave waveform. It has been determined that endothelial cells exposed to such a field will experience an up-regulation in VEGF expression as measured by mRNA production in the cells.

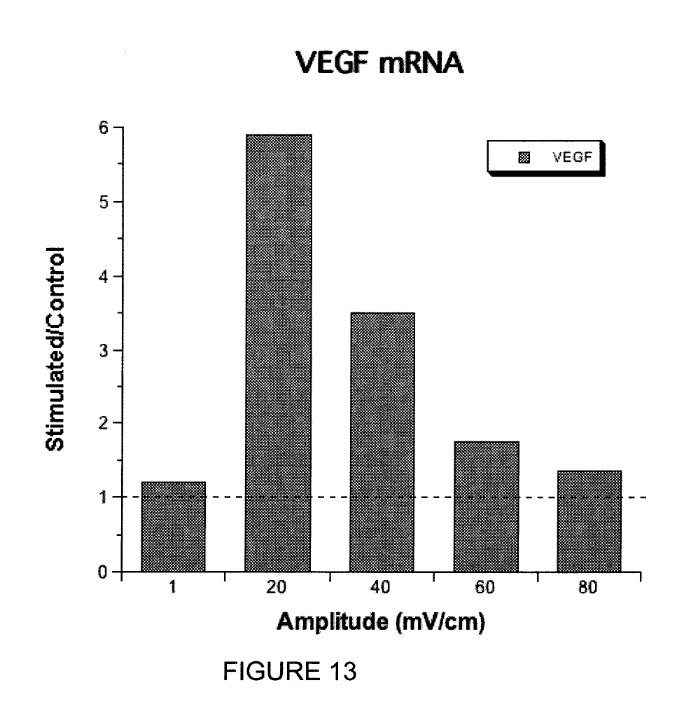

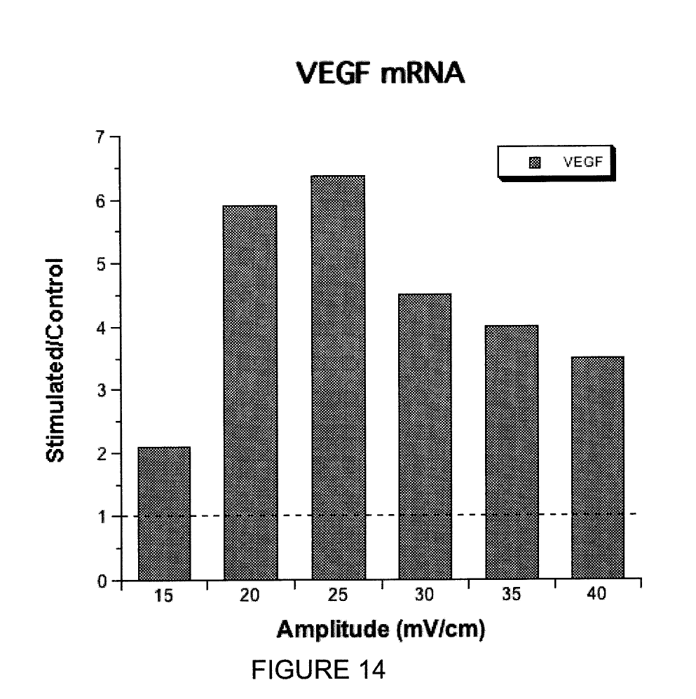

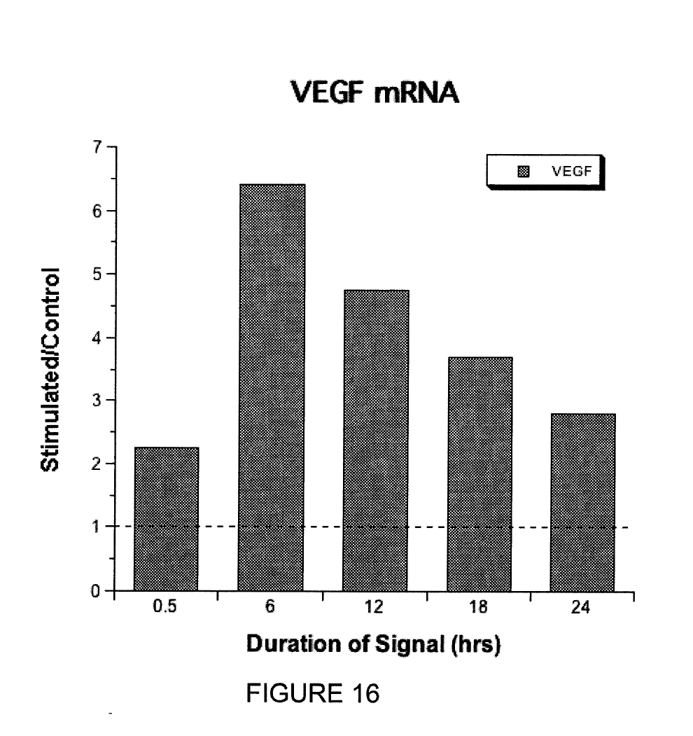

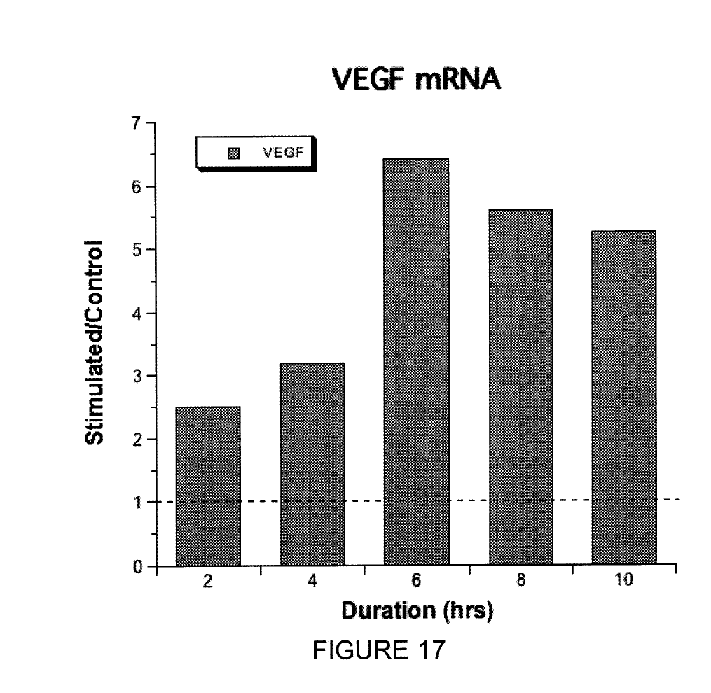

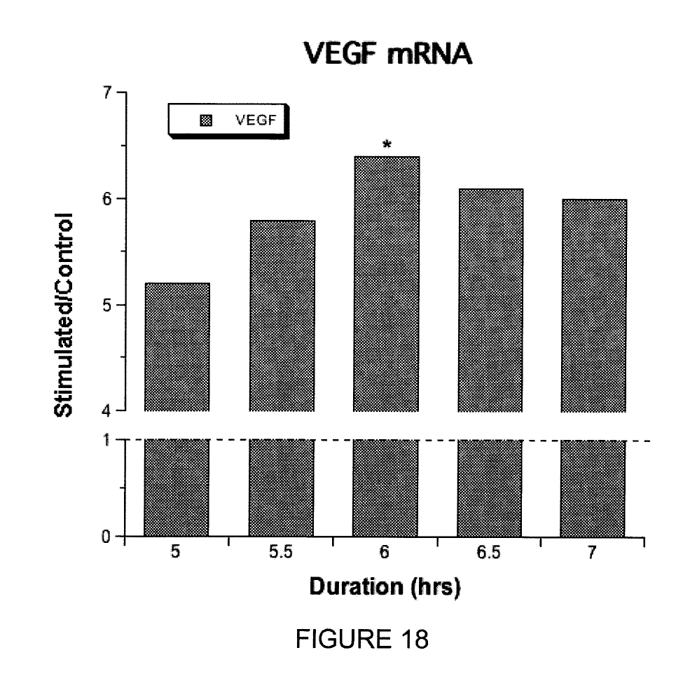

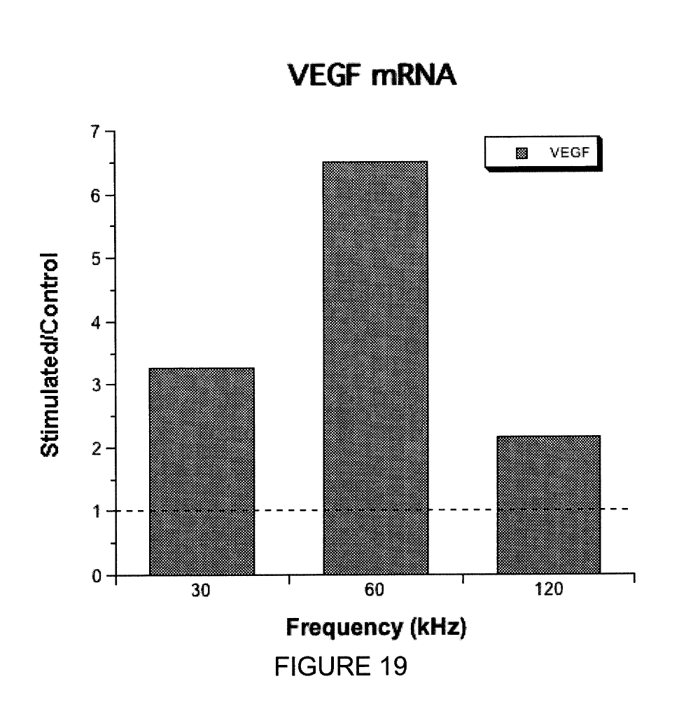

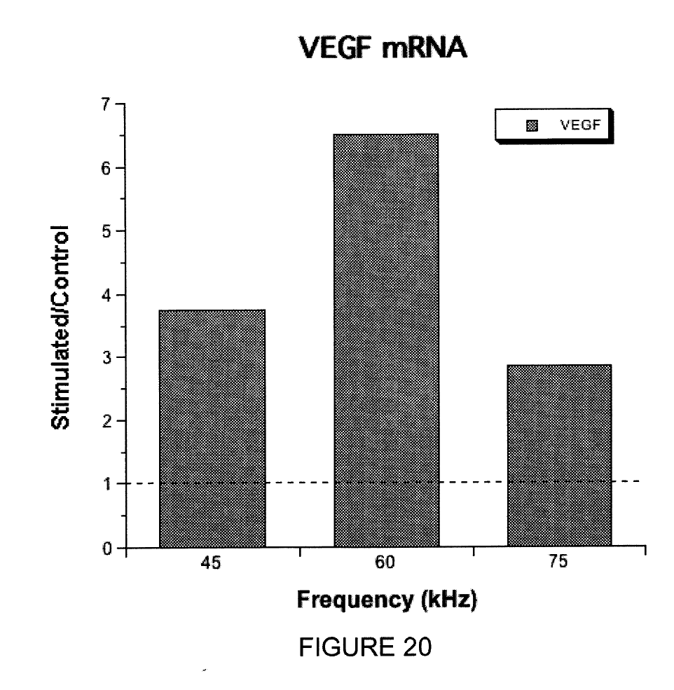

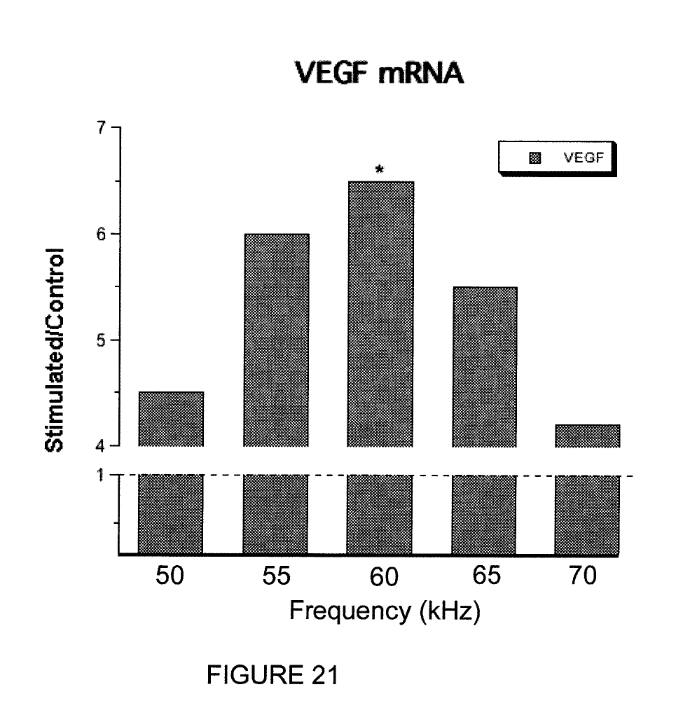

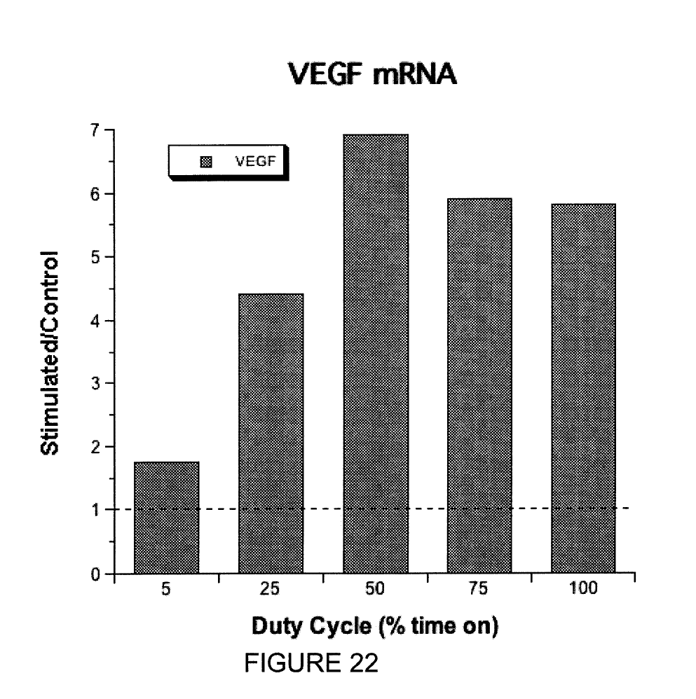

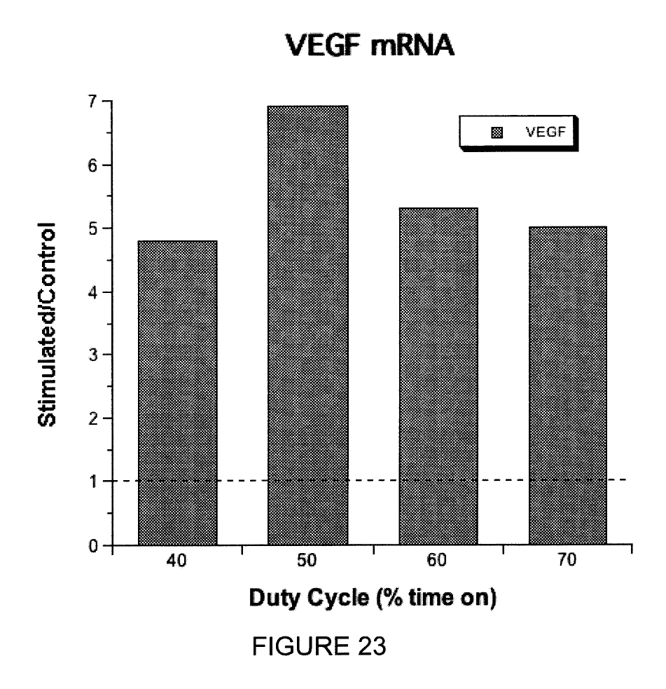

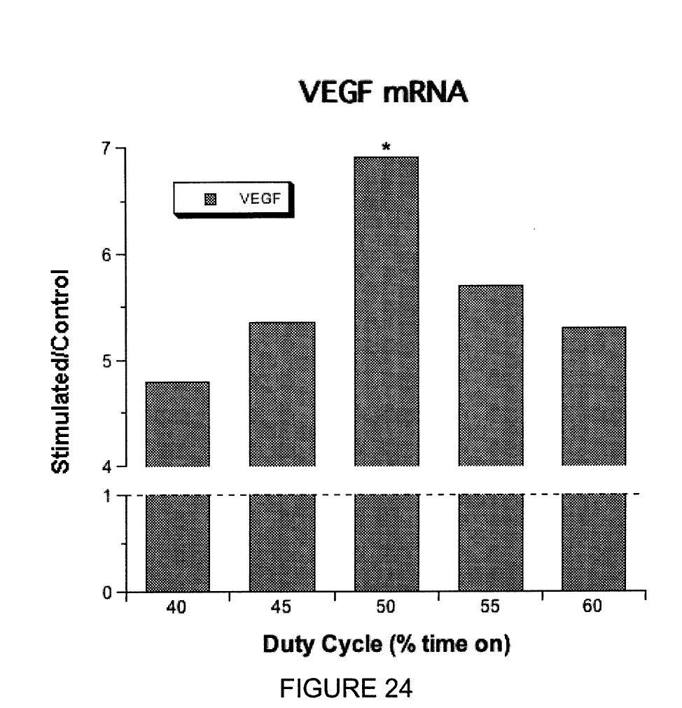

It has also been determined that, based on FIGS. 13-24, and specifically based on those determinations illustrated in FIGS. 14, 17, 20, and 23, regarding the results of performing sequential dose-response curves on characteristics (e.g., duration, amplitude, frequency, duty cycle, and/or waveform) of a signal generating a field to which endothelial cells are exposed, a signal desirable for generating a field that effectively up-regulates VEGF expression in endothelial cells as measured by mRNA production generates a capacitively coupled electric field with an amplitude of between 15 and 40 mV/cm inclusively, a duration of between 2 and 10 hours inclusively, a frequency of between 45 and 75 kHz inclusively, and a duty cycle of between 40 and 70% inclusively, with a sine wave waveform. It has been determined that endothelial cells exposed to such a field will experience a greater increase (as compared with the preferable signal discussed above) up-regulation in VEGF expression as measured by mRNA production in the cells.

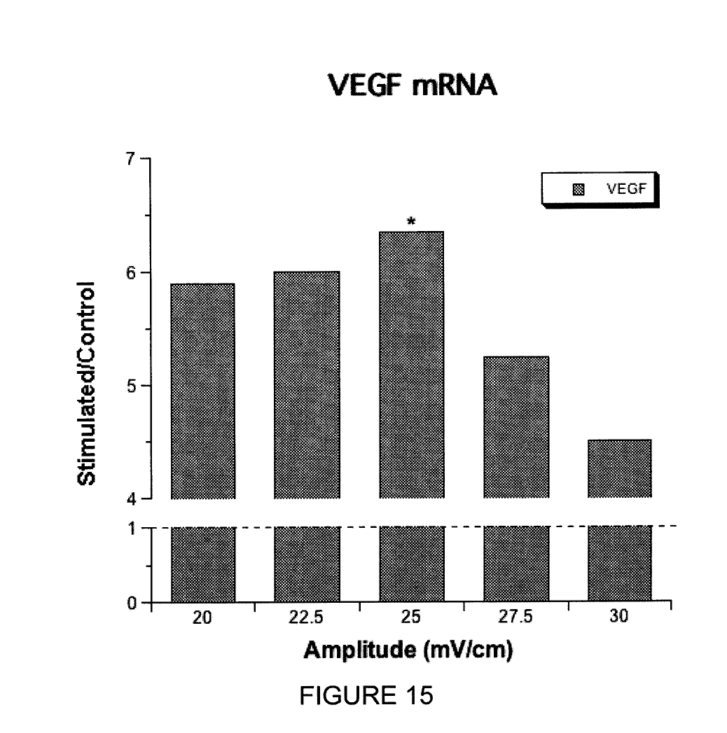

It has been determined that, based on FIGS. 13-24, and specifically based on those determinations illustrated in FIGS. 15, 18, 21, and 24, regarding the results of performing sequential dose-response curves on characteristics (e.g., duration, amplitude, frequency, duty cycle, and/or waveform) of a signal generating a field to which endothelial cells are exposed, a signal desirable for generating a field that effectively up-regulates VEGF expression in endothelial cells as measured by mRNA production generates a capacitively coupled electric field with an amplitude of between 20 and 30 mV/cm inclusively, a duration of between 5 and 7 hours inclusively, a frequency of between 50 and 70 kHz inclusively, and a duty cycle of between 40 and 60% inclusively, with a sine wave waveform. It has been determined that endothelial cells exposed to such a field will experience an even greater increase (as compared with the more preferable signal discussed above) up-regulation in VEGF expression as measured by mRNA production in the cells.

It has also been determined that, based on FIGS. 13-24, and specifically based on those asterisked determinations illustrated in FIGS. 15, 18, 21, and 24, regarding the results of performing sequential dose-response curves on characteristics (e.g., duration, amplitude, frequency, duty cycle, and/or waveform) of a signal generating a field to which endothelial cells are exposed, a signal determined to be desirable for generating a field that effectively up-regulates VEGF expression in endothelial cells as measured by mRNA production generates a capacitively coupled electric field with an amplitude of 25 mV/cm, a duration of 6 hours, a frequency of 60 kHz, and a duty cycle of 50%, with a sine wave waveform. It has been determined that endothelial cells exposed to such a field will experience a desirable amount of up-regulation in VEGF expression as measured by mRNA production in the cells.

### Example 10

### VEGF Production in Endothelial Cells

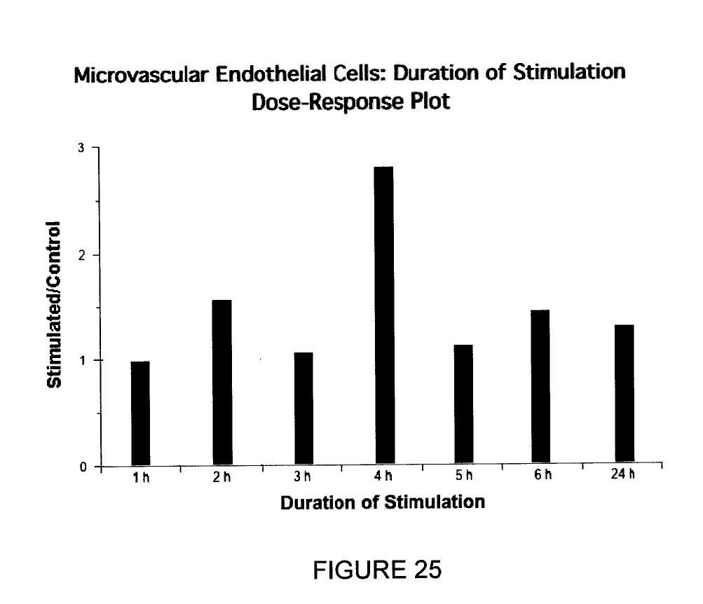

With reference to FIG. 25, in another set of experiments, a signal determined to be desirable for generating a field that effectively up-regulates VEGF expression in endothelial cells as measured by mRNA production generates a capacitively coupled electric field with an amplitude of about 20 mV/cm, a frequency of about 60 kHz, a duty cycle of about 100%, a sine wave waveform, and a duration of between about 3-5 hours, preferably 4 hours. A duration of 4 hours was found to produce superior up-regulation of VEGF expression (almost 3 times) as compared with other durations, e.g., 1 hour, 2 hours, 3 hours, 5 hours, 6 hours and 24 hours.

### Example 11

### VEGF Production in Endothelial Cells

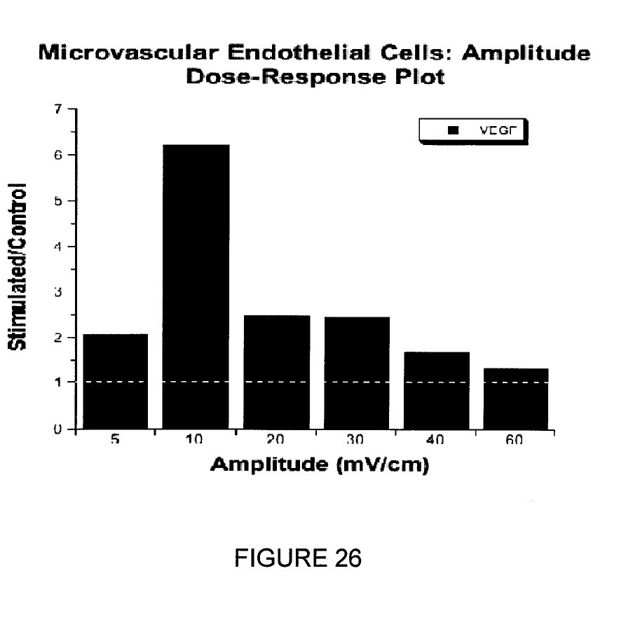

With reference to FIG. 26, in another set of experiments, a signal determined to be desirable for generating a field that effectively up-regulates VEGF expression in endothelial cells as measured by mRNA production generates a capacitively coupled electric field with a frequency of about 60 kHz, a duty cycle of about 100%, a sine wave waveform, a duration of between about 3-5 hours, and an amplitude of about 5-20 mV/cm, preferably 10 mV/cm. An amplitude of about 10 mV/cm was found to produce superior up-regulation of VEGF expression (greater than 6 times) as compared with other amplitudes, e.g., 5 mV/cm, 20 mV/cm, 30 mV/cm, 40 mV/cm, and 60 mV/cm.

### Example 12

### Treatment of Peripheral Vascular Disease

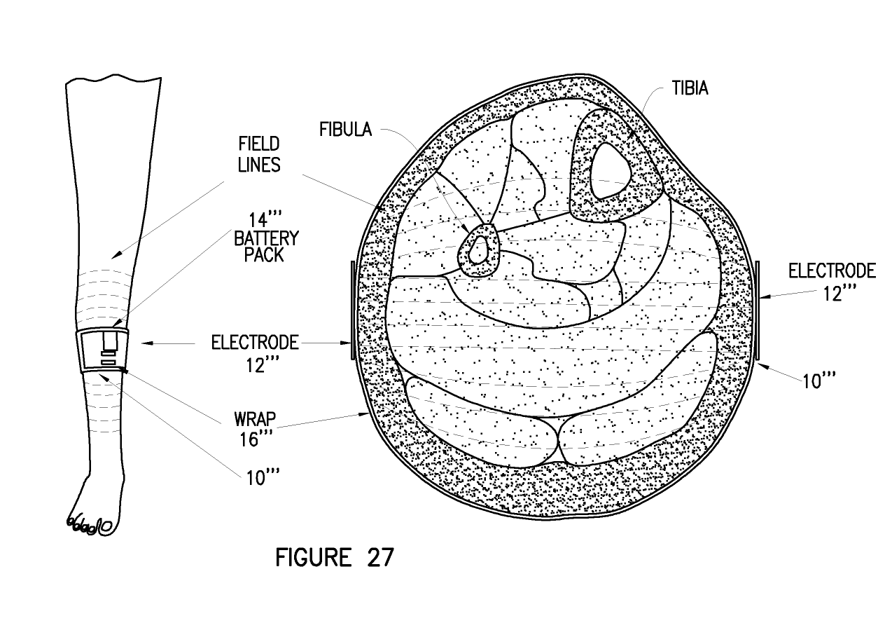

With reference to FIG. 27, a device 10′″ in accordance with preferred embodiments of the present invention is used to treat a patient with peripheral vascular disease of the calf. As illustrated, two circular, soft conductive, self-adherent electrodes 12′″ are placed on the skin on either side of the calf. The electrodes 12′″ are attached to a power unit 14′″ which has a VELCRO® patch 16′″ on the reverse side such that the power unit 14′″ can be attached to a VELCRO® strap (not shown) fitted around the calf. The electrodes 12′″ may be placed on the skin before the patient goes to bed each evening or any other time.

The power unit is preferably small (e.g., 6-8 ounces) and powered by a standard 9-volt battery to emit a signal to the electrodes 12 placed on the skin. As illustrated in Example 9, it has been determined that a signal with characteristics in the preferred ranges, in the more preferred ranges, in the most preferred ranges, or at the optimal settings, will up-regulate VEGF expression in endothelial cells. This treatment should prevent or minimize further vascular deterioration in the calf, as well as reverse the effects of vascular disease.

Those skilled in the art will also appreciate that numerous other modifications to the invention are possible within the scope of the invention. For example, genes encoding for tissue inhibitors of metalloproteinase (“TIMP”) and other genes may have improved specific dose responses at selective frequencies other than 60 kHz so as to provide specific and selective responses for applied signals at different frequencies with different time durations, field amplitudes, and duty cycles. Also, as noted above, inductively coupled signals, direct coupled signals, and pulsed electromagnetic fields may also be applied in lieu of capacitively coupled signals as described in the examples above. Accordingly, the scope of the invention is not intended to be limited to the preferred embodiments described above.

1. A method of up-regulating the gene expression of Vascular Endothelial Growth Factor (VEGF) protein in targeted tissue, comprising the steps of:
a. generating at least one electric signal that when applied to electrodes, one or more coils, or other field generating devices operatively disposed with respect to said targeted tissue causes the generation of an electric field in said targeted tissue that substantially up-regulates the gene expression of VEGF protein in said targeted tissue as measured by mRNA production therein; and
b. exposing said targeted tissue to the electric field generated by said electrodes, one or more coils, or other field generating devices upon application of said at least one electric signal thereto for a predetermined duration of time at predetermined intervals so as to selectively up-regulate the gene expression of VEGF protein in said targeted tissue as measured by mRNA production therein.

2. The method of claim 1, wherein the generating step comprises the step of selectively varying at least one of the amplitude, duration, duty cycle, frequency, and waveform of the applied electric signal until the gene expression of VEGF protein in said targeted tissue as a result of exposure to the resultant electric field as measured by mRNA production in the targeted tissue is substantially increased.

3. The method of claim 1, wherein the exposing step comprises the step of exposing said targeted tissue to the electric field for a duration of between 30 minutes and 24 hours, every 24 hours.

4. The method of claim 1, wherein the exposing step comprises the step of exposing said targeted tissue to the electric field for a duration of between about 3 and 5 hours.

5. The method of claim 4, wherein the duration is about 4 hours.

6. The method of claim 1, wherein the generating step comprises the step of generating an electric signal having a sine wave configuration, a duty cycle of between 1 and 100%, and a frequency of between 30 and 120 kHz and the resultant electric field in the targeted tissue has an amplitude of between 1 and 80 mV/cm.

7. The method of claim 1, wherein the generating step comprises the step of generating an electric signal having characteristics such that the resultant electric field in the targeted tissue has an amplitude of between about 5 and 20 mV/cm.

8. The method of claim 7, wherein the resultant electric field in the targeted tissue has an amplitude of about 10 mV/cm.

9. The method of claim 1, wherein said generating step comprises the step of generating the electric signal at a remote source and said exposing step comprises the step of applying the electric field to the targeted tissue.

10. The method of claim 9, wherein the exposing step comprises the step of applying the electric signal to electrodes, one or more coils, or other field generating devices located near the targeted tissue.

11. The method of claim 10, wherein the exposing step comprises the step of applying the electric field in the targeted tissue generated by the electrodes, one or more coils, or other field generating devices upon application of said at least one electric signal thereto to the targeted tissue through capacitive coupling or inductive coupling.

12. The method of claim 11, wherein the electric signal applied to said electrodes causes the electrodes to generate a capacitive coupling electric field, and the electric signal applied to said one or more coils causes said one or more coils to generate an electromagnetic field or a combined field.

13. A method for treating at least one of peripheral vascular disease, cardiovascular disease, macular degeneration, wound healing, tendon healing, ligament healing, tumor growth, tumor spread, and other conditions in which Vascular Endothelium Growth Factor (VEGF) protein has been implicated in a patient, comprising the steps of:
a. generating at least one electric signal that when applied to electrodes, one or more coils, or other field generating devices operatively disposed with respect to targeted tissue causes the generation of an electric field in the targeted tissue that substantially up-regulates the gene expression of VEGF protein in said targeted tissue as measured by mRNA production; and
b. exposing said targeted tissue to the electric field generated by said electrodes, one or more coils, or other field generating devices upon application of said at least one electric signal thereto for a predetermined duration of time at predetermined intervals so as to selectively up-regulate the gene expression of VEGF protein in said targeted tissue as measured by mRNA production.

14. The method of claim 13, wherein the exposing step comprises the step of capacitively coupling or inductively coupling the electric field to the targeted tissue.

15. The method of claim 13, wherein the exposing step comprises the step of applying one of an electromagnetic field and a combined field to the targeted tissue.

16. The method of claim 13, wherein the generating step comprises the step of generating an electric signal having a sine wave configuration, a duty cycle of between 1 and 100%, and a frequency of between 30 and 120 kHz and the resultant electric field has an amplitude of between 1 and 80 mV/cm in the targeted tissue.

17. The method of claim 13, wherein the exposing step comprises the step of applying the electric field to the targeted tissue for a duration of between 30 minutes and 24 hours at an interval of between 30 minutes and 24 hours.

18. The method of claim 13, wherein the exposing step comprises the step of exposing said targeted tissue to the electric field for a duration of between about 3 and 5 hours.

19. The method of claim 18, wherein the duration is about 4 hours.

20. The method of claim 13, wherein the generating step comprises the step of generating an electric signal having characteristics such that the resultant electric field in the targeted tissue has an amplitude of between about 5 and 20 mV/cm.

21. The method of claim 20, wherein the resultant electric field in the targeted tissue has an amplitude of about 10 mV/cm.

22. The method of claim 13, wherein the generating step comprises the steps of starting with any electric signal that when applied to said electrodes, one or more coils, or other field generating devices generates and electric field that is known or thought to be effective on living cells, performing a first dose-response curve on the amplitude of stimulation of the electric field to determine an optimal amplitude; performing a second dose-response curve on the duration of the applied electric signal using the optimal amplitude as previously found to determine an optimal duration; performing a third dose-response curve on the frequency of the applied electric signal keeping the optimal amplitude and optimal duration as previously found to determine an optimal frequency; performing a fourth dose-response curve varying the duty cycle of the applied electric signal and keeping the optimal duration, amplitude, and frequency as previously found to determine an optimal duty cycle, and keeping the optimal duration, amplitude, frequency and duty cycle constant while varying the waveform until an optimal waveform for the up-regulation of the gene expression of VEGF protein as measured by mRNA production in the targeted tissue is found.

23. A device for the treatment of at least one of peripheral vascular disease, cardiovascular disease, macular degeneration, wound healing, tendon healing, ligament healing, tumor growth, tumor spread, and other conditions in which Vascular Endothelium Growth Factor (VEGF) protein has been implicated in a patient, comprising:
a signal source that generates at least one electric signal; and
electrodes, one or more coils, or other field generating devices that are operatively disposed with respect to targeted tissue, said electrodes, one or more coils, or other field generating devices upon receipt of said at least one electric signal causing the generation of an electric field in the targeted tissue that substantially up-regulates the gene expression of VEGF protein in said targeted tissue as measured by mRNA production upon application of said at least one electric field thereto for a predetermined duration of time at predetermined intervals.

24. The device of claim 23, wherein the signal source is programmable such that fields generated during various modes can be sequentially applied to the targeted tissue for various periods of time and in various orders.

25. The device of claim 23, further comprising means for attaching the electrodes, coils, or other field generating devices to the body of a patient in the vicinity of bone tissue.

26. The device of claim 23, further comprising means for attaching the signal source to the body of a patient.

27. The device of claim 23, wherein the electric field generated by application of said at least one electric signal to the electrodes, coils, or other filed generating devices is applied to said targeted tissue via capacitive coupling or inductive coupling.

28. The device of claim 23, wherein the electric signal has a sine wave configuration a duty cycle of between 1 and 100%, and a frequency of between 30 and 120 kHz and the resultant electric field has an amplitude of between 1 and 80 mV/cm in the targeted tissue.

29. The device of claim 23, wherein the signal source is operable to produce the electric field for a duration of between about 3 and 5 hours.

30. The device of claim 29, wherein the duration is about 4 hours.

31. The device of claim 23, wherein the signal source is operable to generate an electric signal having characteristics such that the resultant electric field in the targeted tissue has an amplitude of between about 5 and 20 mV/cm.

32. The device of claim 31, wherein the resultant electric field in the targeted tissue has an amplitude of about 10 mV/cm.

33. A method of treating at least one of peripheral vascular disease, cardiovascular disease, macular degeneration, wound healing, tendon healing, ligament healing, tumor growth, tumor spread, and other conditions in which VEGF protein has been implicated in a patient, comprising the steps of exposing targeted tissue to the electric field generated by the device of claim 20 so as to up-regulate gene expression of VEGF protein in the targeted tissue as measured by mRNA production in the targeted tissue.

34. A method of determining one or more characteristics of an electric signal that when applied to an electrode, one or more coils or other field generating device causes the generation of an electric field in targeted tissue that up-regulates Vascular Endothelial Growth Factor (VEGF) protein in the targeted tissue as measured by mRNA production, comprising the steps of starting with a starting electric signal with a signal shape and frequency that when applied to said electrodes, one or more coils, or other field generating devices generates an electric field that is known or thought to affect cellular production of VEGF protein, selectively varying a duration of application of said starting signal until a duration that provides a most significant increase in production of VEGF protein is found, selectively varying an amplitude of the starting signal until an amplitude that provides a most significant increase in production of VEGF protein is found, selectively varying a duty cycle of the starting signal until a duty cycle that provides a most significant increase in production of VEGF protein is found, and selectively varying an on-off interval of the duty cycle of the signal until an on-off interval that provides a most significant increase in production of VEGF protein is found.

35. A method as in claim 34, comprising the further steps of selectively varying a frequency and waveform of said starting signal, keeping other signal characteristics constant, until a most significant increase in production of VEGF protein as measured by mRNA production is found.
